(19) United States
(12) Patent Application Publication
(10) Pub. No.: US 2024/0181026 A1
(43) Pub. Date: Jun. 6, 2024

(54) IMMUNOGENIC COMPOSITIONS

(71) Applicant: MACFARLANE BURNET INSTITUTE FOR MEDICAL RESEARCH AND PUBLIC HEALTH LIMITED, Melbourne, Victoria (AU)

(72) Inventors: James Beeson, Melbourne (AU); Gaoqian Feng, Melbourne (AU); Liriye KURTOVIC, Melbourne (AU)

(21) Appl. No.: 18/019,403

(22) PCT Filed: Aug. 6, 2021
(86) PCT No.: PCT/AU2021/050864

§ 371 Date: Feb. 2, 2023

(30) Foreign Application Priority Data
Aug. 6, 2020 (AU) 2020902779

(51) Int. Cl. A61K 39/015; A61K 39/00; A61P 33/06

(52) U.S. Cl. CPC A61K 39/015; A61P 33/06; A61K 2039/5258; A61K 2039/55555

(57) ABSTRACT

Immunogenic or vaccine compositions for preventing malaria, comprising or encoding CSP N-terminal (NT) sequences capable of presenting NT epitopes to a subject, and methods of administering same.

### FIELD

This disclosure relates to the technical field of malaria vaccines.

### BACKGROUND

The reference in this specification to any prior publication, or to any matter which is known, is not, and should not be taken as an acknowledgment or admission or any form of suggestion that that prior publication or known matter forms part of the common general knowledge in the field of endeavour to which this specification relates.

Bibliographic details of documents referred to are listed at the end of the specification.

Malaria remains a major global health concern, with Plasmodium. falciparum malaria causing approximately 219 million clinical cases and 435,000 deaths annually. Despite international control efforts, the malaria burden has remained constant in recent years. There is a strong need for a highly efficacious vaccine for malaria control and elimination, which is further emphasised by increasing reports of antimalarial drug resistance and insecticide resistance. The World Health Organization and funding partners set a goal to develop a vaccine with more than 75% efficacy by 2030, but this has proven challenging to achieve. To improve vaccine efficacy, a greater understanding of the mechanisms that mediate immunity is needed to develop strategies that generate more potent protective immune responses.

Malaria infection starts from an infected Anopheles mosquito bite, during which sporozoites are inoculated into the dermis. Sporozoites then migrate through to a blood vessel, circulate in the blood stream to the liver, and establish infection in hepatocytes. Sporozoites represent a priority target of malaria vaccines because clearing sporozoites will halt infection prior to the onset of clinical malaria, which occurs during the subsequent blood stage infection. Antibodies are thought to be the main mediator of immunity to sporozoites, as demonstrated in animal models and vaccine trials (Beeson, J. G., et al. Challenges and strategies for developing efficacious and long-lasting malaria vaccines, Sci Transl Med 11(2019)). The circumsporozoite protein (CSP) is the most abundant protein expressed on the surface of sporozoites, and is a major target of human antibodies. Vaccines based on P. falciparum CSP have out-performed other vaccine antigens to date, although efficacy remains sub-optimal. The most advanced malaria vaccine, RTS,S, is based on a truncated form of CSP (containing only the central repeat region and C-terminal region) and achieved modest efficacy against clinical malaria (26-36%) in the phase III clinical trial of infants and young children. The central repeat region of CSP is considered an important antibody target, and consequently, the importance of antibodies to non-repeat regions has not been clarified. Antibodies to the C-terminal region have been associated with protection in RTS,S vaccine trials in children and some C-terminal epitopes are shielded by the N-terminal domain which is absent in the RTS,S VLP. In terms of the functional mechanisms of antibody activities, the focus has been on the direct inhibitory activity of antibodies to the central repeat region of CSP, which can inhibit sporozoite motility and hepatocyte invasion in vitro.

There is a need for more efficacious vaccines to control malaria.

### SUMMARY

The term “and/or”, e.g., “X and/or Y” shall be understood to mean either “X and Y” or “X or Y” and shall be taken to provide explicit support for both meanings or for either meaning.

As used herein, the term “about” in relation of N-terminal fragments or epitope regions thereof, unless stated to the contrary, refers to +/−2 amino acids, or +/−3 amino acid, of the specified peptide fragment.

Throughout this specification the word “comprise”, or variations such as “comprises” or “comprising”, will be understood to imply the inclusion of a stated element, integer or step, or group of elements, integers or steps, but not the exclusion of any other element, integer or step, or group of elements, integers or steps. The terms “protein”, “polypeptide” and “peptide” are used interchangeably herein when referring to a gene product.

The terms “subject,” “mammal,” and “patient” are used interchangeably. In some embodiments, the subject is a mammal. In some embodiments, the mammalian subject is a human. In some embodiments, the subject is a mouse, rat, rabbit, dog, donkey, non-human primate, or a laboratory test animal such as fruit fly, zebrafish, etc.

It is contemplated that the methods and compositions may include exclusion of any of the embodiments described herein.

The term “substantially” is defined as being largely but not necessarily wholly what is specified (and include wholly what is specified) as understood by one of ordinary skill in the art. In any disclosed embodiment, the term “substantially” may be substituted with “within [a percentage] of” what is specified, where the percentage includes 0.1, 1, 5, and 10 percent.

The feature or features of one embodiment may be applied to other embodiments, even though not described or illustrated, unless expressly prohibited by this disclosure or the nature of the embodiments.

As used herein, the singular form “a”, “an” and “the” include singular and plural references unless the context indicates otherwise.

Nucleotide and amino acid sequences are referred to by a sequence identifier number (SEQ ID NO:). The SEQ ID NOs: correspond numerically to the sequence identifiers <400>1 (SEQ ID NO:1), <400>2 (SEQ ID NO:2), etc. Sequence identifiers are described in Table 1. A sequence listing is provided after the claims. The sequence listing named as 531124PCT filed 6 Aug. 2021 is also incorporated herein by reference.

In one aspect, this disclosure provides immunogenic or vaccine compositions for preventing malaria, comprising or encoding CSP N-terminal (NT) sequences capable of presenting NT epitopes to a subject, and methods of administering same.

Accordingly, in some embodiments, the present invention provides and enables a method of inducing in a subject antibodies against P. falciparum (Pf) sporozoites for vaccinating the subject. In one embodiment, the method comprises administering to the subject one or more peptide antigens or their encoding sequence representing specific subregions of a circumsporozoite polypeptide (CSP) selected from NT, and CT and/or NANP regions. In one embodiment the method comprises:
(i) administering a peptide or a sequence encoding a peptide presenting N-terminal (NT) epitopes of P/CSP; or
(ii) co-administering the peptide or sequence of (i) and a peptide or a sequence encoding a peptide presenting NANP and/or CT epitopes of PfCSP; or
(iii) administering a peptide or a sequence encoding a peptide representing NT, and CT and/or NANP subregions of PfCSP but not full length PfCSP.

The discovery that NT CSP epitopes as shown herein elicit a functional antibody response and specifically a PCMS response provides methods of screening for vaccine candidates by screening for their ability to engender PCMS responses. Surprisingly, regions of the NT subregion of CSP have been identified that elicit, by themselves strong PCMS responses. In some embodiments, the present invention provides and enables a method of inducing in a subject antibodies against P. falciparum (Pf) sporozoites for vaccinating the subject comprising administering to the subject one or more peptide antigens, or their encoding sequence, representing specific subregions of a circumsporozoite polypeptide (CSP) including NT by itself, the method comprises:
(i) administering a peptide or a sequence encoding a peptide presenting N-terminal (NT) epitopes of PfCSP.

In some embodiments, the present invention provides and enables a method of inducing in a subject antibodies against P. falciparum (Pf) sporozoites for vaccinating the subject comprising administering to the subject one or more peptide antigens, or their encoding sequence, representing specific subregions of a circumsporozoite polypeptide (CSP) including NT by itself or NT and CT and/or NANP subregions, the method comprises:
(i) administering a peptide or a sequence encoding a peptide presenting N-terminal (NT) epitopes of PfCSP; or
(ii) co-administering the peptide or sequence of (i) and a peptide or a sequence encoding a peptide presenting NANP and/or CT epitopes of PfCSP.

In one embodiment, the present invention provides and enables a method of inducing in a subject antibodies against P. falciparum (Pf) sporozoites for vaccinating the subject comprising administering to the subject one or more peptide antigens, or their encoding sequence, representing specific subregions of a circumsporozoite polypeptide (CSP) and specifically NT and CT and/or NANP subregions, the method comprises: (iii) administering a peptide or a sequence encoding a peptide representing NT, and CT and/or NANP subregions of PfCSP but not full length PfCSP.

Accordingly, in some embodiments, the present invention provides and enables a method of inducing in a subject antibodies against P. falciparum (Pf) sporozoites for vaccinating the subject. In one embodiment, the method comprises administering to the subject one or more peptide antigens representing specific subregions of a circumsporozoite polypeptide (CSP) selected from NT, and CT and/or NANP regions.

For, clarity, in one embodiment the method comprises:
(i) administering a peptide or a sequence encoding a peptide presenting N-terminal (NT) epitopes of PfCSP and not representing NANP or CT subregions of PfCSP; or
(ii) co-administering the peptide or encoding sequence of (i) and a peptide or a sequence encoding a peptide presenting NANP and/or CT epitopes of PfCSP; or
(iii) administering a peptide or a sequence encoding a peptide presenting NT, and CT and/or NANP subregions of PfCSP but not full length PfCSP.

In one embodiment, full length CSP may be absent a signal sequence or other terminal regions for purposes of maximising expression in vitro.

In one embodiment, the NT subregion of CSP is the full length NT region, or the full length NT subregion lacking the signal sequence or amino acids 58 to 104 (SEQ ID NO: 1) of the P. falciparum CSP and the peptide presenting NT epitopes of SEQ ID NO: 1 comprises 3 to 48 contiguous amino acids from the N-terminal amino acids 58 to 104 (SEQ ID NO: 1) of CSP of strain 3D7, or a corresponding peptide from a different P. falciparum strain.

In one embodiment, the NT subregion of CSP is amino acids 58 to 81 (SEQ ID NO: 2) of the P. falciparum CSP and the peptide presenting NT epitopes of SEQ ID NO: 2 comprises 3 to 24 contiguous amino acids of SEQ ID NO: 2 of strain 3D7 or a corresponding peptide from a different P. falciparum strain.

In one embodiment, the NT subregion is amino acids 64 to 84 (SEQ ID NO: 3) of the PfCSP and the peptide presenting NT epitopes of SEQ ID NO: 3 comprises 3 to 21 contiguous amino acids of SEQ ID NO: 3 or a corresponding peptide from a different strain.

Reference to 3 to 48, 3 to 24 or 3 to 21, includes for example 6 to 48, 6 to 24 and 6 to 21, and 12 to 48, 12 to 24, 12 to 21, and 15 to 48, 15 to 24 and 15 to 21. The skilled person is provided with assays herein to confirm the PCMS responses of peptides.

In one embodiment, the peptide representing NT epitopes comprises 3 to 24 contiguous amino acids from the N-terminal amino acids 58 to 81 (SEQ ID NO: 2) of CSP or a corresponding sequence from a different strain, and additionally comprises 3 to 24 contiguous amino acids from the N-terminal amino acids 82 to 104 (SEQ ID NO: 4) of CSP or a corresponding peptide from a different strain.

In one embodiment, wherein the peptide presenting CSP NT epitopes comprises 3 to 21 contiguous amino acids from the N-terminal amino acids 64 to 84 of the CSP polypeptide wherein at least 3 contiguous amino acids include DDG or GNN, and wherein the peptide and additionally comprises 3 to 24 contiguous amino acids from the N-terminal amino acids 76 to 100 (SEQ ID NO: 5) of the CSP polypeptide wherein at least 3 contiguous amino acids include KPK and not GNP.

In one embodiment, wherein the peptide presenting CSP NT epitopes comprises an amino acid sequence selected from one or more of SEQ ID NO: 1 to SEQ ID NO: 39, or SEQ ID NO: 57.

In one embodiment, the peptide presenting CSP NT epitopes comprises a T-cell helper epitope.

In one embodiment, the peptide representing CSP NT epitopes comprises a heterologous T-cell helper epitope.

In one embodiment, the peptide presenting CSP NANP or CT epitopes of PfCSP is RTS,S or R21 vaccine peptide.

In one embodiment, a peptide representing NT, CT and NANP subregions of PfCSP comprises amino acids 59 to 327 or 60 to 327 of CSP (SEQ ID. NO:6) QENWYSLKKNSRSLGENDDGNNEDNEKLRKPKHKKLKQPADGNPDPNANPN VDPNANPNVDPNANPNVDPNANPNANPNANPNANPNANPNANPNANPNANP NANPNANPNANPNANPNANPNANPNANPNANPNANPNVDPNANPNANPNAN PNANPNANPNANPNANPNANPNANPNANPNANPNANPNANPNANPNANPNA NPNANPNANPNKNNQGNGQGHNMPNDPNRNVDENANANSAVKNNNNEEPS DKHIKEYLNKIQNSLSTEWSPCSVTCGNGIQVRIKPGSANKPKDELDYANDIEK KICKMEKCSSVFNVVNSGS, or a corresponding sequence from a different Pf strain.

In one embodiment, a peptide presenting NT epitopes comprises ENWYSLKKNSRSLGENDDGNNEDNEKLRKPKHKKLKQPADG (SEQ ID NO: 57) or ENWYSLKKNSRSLGENDDGNNEDNEKLRKPKHKKLKQPADSGSGQYIKANSK FIGITEL (SEQ ID NO: 60.

In one embodiment, the antigen/s are administered in protein and/or nucleic acid form, as viral like particles, together with nanocarriers/liposomes and/or in pharmaceutical compositions comprising an adjuvant or immunomodulatory agent. Peptide antigens may be administered in protein form (eg, expressed or synthetic antigen) and/or nucleic acid form (eg. mRNA and DNA), as viral like particles or other nanoparticles, together with nanocarriers/liposomes, viral vectors, and/or in pharmaceutical compositions comprising an adjuvant or immunomodulatory agent.

In another aspect the present invention provides and enables a pharmaceutical composition comprising a peptide antigen or antigen encoding sequence representing the NT subregion of CSP SEQ ID NO: 1, wherein the antigen comprises a peptide that presents NT epitopes of SEQ ID NO: 1 when administered to a subject and comprises 3 to 48 contiguous amino acids of SEQ ID NO: 1, or a corresponding peptide from a variant P. falciparum strain, and a pharmaceutically acceptable excipient or diluent.

In one embodiment of the composition, the antigen comprises a heterologous spacer or immunomodulatory element, in one embodiment a helper T-cell epitope.

In one embodiment of the composition, the CSP NT peptide comprises SEQ ID comprises DDGNNEDNEKLRKPKHKKLKQ (SEQ ID NO: 25) or ENWYSLKKNSRSLGENDDGNNEDNEKLRKPKHKKLKQPADG (SEQ ID NO: 57) or KQENWYSLKKNSRSLGENDDGNNEDNEKLRKPKHKKLKQPADGNPDP (SEQ ID NO: 1), or wherein the peptide comprises SEQ ID NO:25 and N-terminal or C-terminal contiguous amino acids from the CSP NT sequences, SEQ ID NO: 57 or SEQ ID NO: 1, or a corresponding sequence from a variant strain.

In one embodiment the pharmaceutical composition further comprises an antigen peptide or a sequence encoding a peptide antigen representing NANP and or CT subregions of PfCSP and presenting NANP and/or CT epitopes of PfCSP.

In another embodiment, a viral like particle composition is enables wherein the viral like particle comprises a peptide antigen representing the NT subregion of CSP SEQ ID NO: 1, wherein the antigen comprises a peptide that presents NT epitopes of SEQ ID NO: 1 when administered to a subject and comprises 3 to 48 contiguous amino acids of SEQ ID NO: 1, or a corresponding peptide from a variant P. falciparum strain, and a pharmaceutically acceptable excipient or diluent. In one embodiment, the antigen comprises a heterologous spacer or immunomodulatory element, in one embodiment a helper T-cell epitope.

In one embodiment of the VLP composition, the CSP NT peptide comprises SEQ ID comprises DDGNNEDNEKLRKPKHKKLKQ (SEQ ID NO: 25) or ENWYSLKKNSRSLGENDDGNNEDNEKLRKPKHKKLKQPADG (SEQ ID NO: 57) or KQENWYSLKKNSRSLGENDDGNNEDNEKLRKPKHKKLKQPADGNPDP (SEQ ID NO: 1), or wherein the peptide comprises SEQ ID NO:25 and N-terminal or C-terminal contiguous amino acids from the CSP NT sequences, SEQ ID NO: 57 or SEQ ID NO: 1, or a corresponding sequence from a variant strain.

In another embodiment, the present description enables the skilled person to make and use a vector or polynucleotide encoding and capable of expressing an antigen as defined herein. In one embodiment, the peptide antigen represents the NT subregion of CSP SEQ ID NO: 1, wherein the antigen comprises a peptide that presents NT epitopes of SEQ ID NO: 1 when administered to a subject and comprises 3 to 48 contiguous amino acids of SEQ ID NO: 1, or a corresponding peptide from a variant P. falciparum strain, and a pharmaceutically acceptable excipient or diluent. In one embodiment, the antigen comprises a heterologous spacer or immunomodulatory element, in one embodiment a helper T-cell epitope.

In one embodiment of the encoded CSP NT peptide comprises SEQ ID comprises DDGNNEDNEKLRKPKHKKLKQ (SEQ ID NO: 25) or ENWYSLKKNSRSLGENDDGNNEDNEKLRKPKHKKLKQPADG (SEQ ID NO: 57) or KQENWYSLKKNSRSLGENDDGNNEDNEKLRKPKHKKLKQPADGNPDP (SEQ ID NO: 1), or wherein the encoded peptide comprises SEQ ID NO:25 and N-terminal or C-terminal contiguous amino acids from the CSP NT sequences, SEQ ID NO: 57 or SEQ ID NO: 1, or encodes a corresponding sequence from a variant strain. In another embodiment, the present application provides a viral or non-viral vector comprising a nucleic acid sequence encoding a peptide antigen representing or comprising NT epitopes of PfCSP and optionally substantially not representing or comprising CSP CT and/or NANP epitopes.

In another aspect the present invention provides a method of treating or preventing malaria comprising administering to a subject an effective amount of a composition as defined herein, a VLP as defined herein, or a vector or polynucleotide as defined herein.

In one aspect there is provided a use of a PCMS antigen or NT CSP peptide antigen as defined herein, or a nucleic acid molecule encoding same, in the manufacture of a medicament for treating or preventing a Plasmodium infection or malaria in a subject.

In one embodiment, the description provides a method of screening vaccine candidate/epitopes comprising testing their ability to bind Fcγ-receptors on neutrophils and monocytes and to induce antibody dependent opsonic phagocytosis of sporozoites or killing by natural killer cells.

### BRIEF DESCRIPTION OF THE FIGURES

The patent or application file contains at least one drawing executed in colour. Copies of this patent or patent application publication with colour drawing(s) will be provided by the Patent Office upon request and payment of the necessary fee.

The accompanying drawings, which are incorporated into and form a part of the specification, illustrate several embodiments of the present invention and, together with the description, serve to explain the principles of the invention.

FIG. 1A-E illustrates the results described in Example 1 showing that neutrophils predominately mediate opsonic phagocytosis of CSP-coated beads and sporozoites in whole blood.

(A) CSP-coated beads were opsonised with different concentrations of rabbit anti-CSP IgG. Neutrophils (left hand side) showed greater phagocytosis activity compared to monocytes (right hand side) (two-way ANOVA P<0.001). Bars represent the mean and standard error from 6 independent experiments.

(B) CSP-coated beads were opsonised with rabbit anti-CSP IgG at 1/500 dilution, and co-incubated at different ratios with whole leukocytes. Phagocytosis activity was substantially higher by neutrophils (LHS) than monocytes (RHS) (two-way ANOVA, P<0.001). Bars represent the mean and standard error from 3 independent experiments.

(C) Neutrophils were tested for phagocytosis of sporozoites using a transgenic P.berghei sporozoite line that expressed CSP of P. falciparum (PfCSP-P.berghei), opsonised with pooled immune sera from immune adults. In the presence of immune antibodies, more sporozoites were phagocytosed by neutrophils (dark blue bar-LHS) than monocytes (light blue bar—RHS-two-way ANOVA, P=0.005). Error bars represent standard error based on data from 2 independent experiments.

(D) The number of beads phagocytosed by each monocyte subset was quantified and standardised as beads per 100 monocytes. The classical monocyte subset showed greater phagocytic activity (P<0.001). Bars and error bars represents the mean and standard error from 5 experiments.

(E) Opsonisation of chimeric PfCSP-P.berghei transgenic sporozoites with a pool of malaria-exposed immune adult sera induced higher level of phagocytosis by neutrophils compared to unopsonised control (P=0.049). Opsonic phagocytosis activity was expressed as the percentage of ethidium bromide-positive cells, and results from two independent experiments are shown (mean and standard error shown).

FIG. 2A-H illustrate the roles of specific Fcγ-receptors in mediating opsonic phagocytosis of sporozoites as described in Example 2, and legend to FIG. 2.

(A) Effect of blocking specific FcγRs on neutrophil phagocytosis of opsonised sporozoites; P. falciparum sporozoites were opsonised with a pool of malaria-exposed adult sera. Bars represent mean and standard error of phagocytosis index based on results from two experiments conducted in duplicate.

(B) Effect of blocking specific FcγRs on phagocytosis of opsonised CSP-beads by neutrophils. CSP-coated beads were opsonised with a pool of serum from Immune adults. Bars represent mean and standard deviation from two experiments conducted in duplicate.

(C) Effect of blocking FcγR receptors on phagocytosis of CSP-beads by THP-1 cells. Bars represent mean and standard error from two experiments conducted in duplicate.

(D) Opsonic phagocytosis of CSP-coated beads by THP-1 cells was much lower in the presence (light blue bars −RHS) compared to absence (dark blue bars LHS) of heat-inactivated human serum (two-way ANOVA, P<0.001). Error bars represent standard error from two independent experiments.

(E) Antibodies from selected immune serum samples induced ADCC activity using CSP-coated beads and an ADCC reporter cell line. Selected individuals (AR1 to AR13) are shown as examples to demonstrate the variation of activity observed. Sera from malaria naïve Melbourne donors were used as negative controls (MC)

(F) Correlation between antibody activity in FcγRIII binding and ADCC assays (reporter cell line) among immune adult serum samples (n=31).

(G) Antibodies from the same immune serum samples were tested for ADCC activity using CSP-coated beads and primary NK cells. Selected individuals (S1 to S13) are shown as representative examples.

(H) Correlation between antibody-mediated FcγRIII binding and ADCC activity (using primary NK cells) among immune adult serum samples (n=31).

* indicates significant difference (p<0.05); ns, not significant, RPI, relative phagocytosis index

FIG. 3A-B illustrate direct quantification of FcγRIIa and FcγRIII binding activity as described in Example 3.

(A) Antibodies to CSP among immune adults were tested for their ability to promote binding to FcγRIIa (LHS) and FcγRIII (RHS). Bars and error bars represent the mean and standard deviation for each sample tested in duplicate. Representative individuals are shown to reflect the range of activities observed.

(B) Correlation between FcγRIIa binding and FcγRIII binding in the immune adult cohort. There was significant correlation between FcγRIIa binding and FcγRIII binding (n=104).

FIG. 4A-E illustrate CSP-specific antibodies induced opsonic phagocytosis by neutrophils as described in Example 4.

(A) Opsonic phagocytosis of P. falciparum sporozoites by neutrophils mediated by rabbit polyclonal antibody to CSP, compared to no antibody (no Ab) control. Data show mean and standard errors from three independent experiments.

(B) Opsonic phagocytosis of P. falciparum sporozoites by neutrophils mediated by mouse monoclonal antibody (2H8) against CSP at different concentration, compared to mouse monoclonal antibody (3D11) against P. berghei CSP as a control. Data shown are mean and standard errors from three independent experiments.

(C) The level of opsonic phagocytosis of beads by neutrophils varied among beads coated with different regions of CSP (P=0.022). Beads coated with full-length CSP (FL), the CT region, the NT region and the NANP repeat region (repeats) were opsonised with antibodies from a pool of immune adult donors. Unopsonised beads were used as negative control for assay background. Data show mean and standard error calculated based on three independent experiments.

(D) Rabbit IgG to the NT region was more efficient in promoting phagocytosis of PfCSP-P. berghei sporozoites by neutrophil compared to Rabbit IgG to the CT region (P=0.0079). Data shown are mean and standard error based on two independent experiments. Phagocytosis activity was standardized relative to the level of IgG to CSP of each rabbit antibody.

(E) Antibodies from the immune adult pool were tested for their ability to promote the binding of FcγRIIa and FcγRIII binding activity with each region of CSP (same orders as for C). The level of FcγR binding was standardized according to the level of total IgG binding to each CSP construct (FcR binding efficiency). Data show mean and standard error based on two independent experiments. Differences in FcγR activity between regions was not significant (p>0.3).

FIG. 5A-C illustrate immunization of rabbits with different CSP regions induced antibodies to CSP

(A) Rabbits immunized with the N+C region of CSP only generated antibodies to the CT region of CSP. Figures show IgG reactivity of antibodies to full length CSP (FL), NT region (NT), central NANP-repeat region (NANP), and CT region (CT), determined by ELISA. (B) Rabbits immunized with the NT region peptide only generated antibodies to the NT region of CSP. (C) The level of IgG reactivity to full length CSP (FL) by rabbit antibodies raised against the NT region and CT region, determined by ELISA. Error bars represent the standard deviation of OD values from two replicates.

FIG. 6 illustrates the gating strategy for whole leukocyte assays and to quantify the level of phagocytosis by neutrophils and monocytes. Cells, which phagocytosed beads, were gated based on fluorescent intensity (FITC^{high}) and size (FCS^{high}). This population was further divided into neutrophils (CD66b^{high}) and monocytes (CD66b^{low} and CD14⁺). The number of beads phagocytosed by neutrophils or monocytes were determined based on the beads fluorescent intensity. These numbers were further standardized according to the number of phagocytes (including neutrophils and monocytes) acquired in each sample and expressed as beads per 100 phagocytes.

FIG. 7 illustrates the gating strategy for ADCC assay using primary NK cells and to quantify the level of ADCC in primary NK cells. The NK cells were defined as CD3⁻ and CD56⁺ lymphocytes. The level of ADCC were determined as percentage of CD107a positive NK cells.

FIG. 8 illustrates the establishment of a phagocytosis assay using THP-1 cells.

(A) Cryo-preserved P. falciparum sporozoites were stained with ethidium bromide, opsonised with a pool of immune adult sera and phagocytosed by THP-1 cells. Pooled serum samples from malaria-naïve Melbourne donors were used as negative control. A higher level of phagocytosis was seen when sporozoites were opsonised with immune adult sera. The level of phagocytosis is presented as phagocytosis index which reflects the percentage of THP-1 cells that have taken up fluorescently stained sporozoites (data show mean and standard error).

(B) Reproducibility of the opsonic phagocytosis assay using cryo-preserved sporozoites and THP1 cells (Spearman's rho=0.925, p<0.01). Cryo-preserved sporozoites were opsonised with a selection of serum samples from malaria-exposed subjects to represent high, intermediate and low responders based on total IgG to CSP. Phagocytosis assays were performed twice independently. Phagocytosis index was standardized to the percentage of the positive control, which was a pool of immune adult serum.

(C) Opsonic phagocytosis activity by THP-1 cells was strongly correlated when using CSP-coated beads or cryo-preserved P. falciparum sporozoites (Spearman's rho=0.831, p<0.01). Cryo-preserved sporozoites or CSP coated beads were opsonised with a selection of serum samples from an immune adult cohort to represent high, intermediate and low responders based on total IgG to CSP. The phagocytosis index was standardized to the percentage of the positive control, which was a pool of immune adult serum and defined as relative phagocytosis index (RPI).

FIG. 9 illustrates the gating strategy for analysis within monocyte subsets.

The monocyte population were further divided into classical monocytes (CD14^{high} CD16⁻), intermediate monocytes (CD14^{high} CD16⁺) and non-classical monocytes (CD1410^{low} CD16⁺).

FIG. 10 illustrates the titration of FcγRIIa and FcγRIII blocking antibodies.

Neutrophils were blocked by FcγRIIa and FcγRIII blocking antibodies at concentration of 100 ul/ml (dark blue bars), 25 μg/ml (light blue bars) and 6.25 μg/ml (cyan blue bars) prior co-incubation of opsonised CSP-coated beads for phagocytosis. No differences were observed when the FcγRs were blocked by blocking antibodies at different concentrations (P>0.1). Bars and error bars represent the mean and standard deviation of each experiment condition tested in duplicates.

FIG. 11 is a graphical representation of results showing the activity of antibodies raised against the NT of CSP. Rabbit antibodies raised against the NT region of CSP were tested for IgG reactivity (left panel) and complement fixation activity (C1q binding; right panel) using immobilised full-length CSP (lower) or N-terminal protein (upper). Results show mean and range from two independent experiments.

FIG. 12 is a graphical representation of data showing vaccination of rabbits with full-length CSP does not effectively generate antibodies to the N-terminal region of CSP (bottom pink line). Data show IgG reactivity (left panel) of antibodies to different regions of CSP (NT, NANP, and CT regions; full-length CSP for comparison) and complement fixation activity (C1q binding; right panel). Mean and range of two independent experiments.

FIG. 13 illustrates the results of epitope mapping of polyclonal rabbit IgG raised against the N-terminus of CSP. Antibodies were tested against an overlapping peptide array derived from the CSP sequence (3D7 strain). N-terminal peptide sequences are listed below. Bars and error bars represents mean and standard error from two independent experiments.

FIG. 14 illustrates how depletion of antibodies to the N-terminal region of CSP among human antibodies decreased FcγRIIa binding efficiency of the antibodies.

Serum antibodies (from different malaria-exposed adults) were incubated with recombinant NT protein to deplete NT antibodies from the pool. The ability of antibodies to promote FcγRIIa binding was then tested with/without NT-antibody depletion and further standardized according to the total IgG titre to full-length CSP with/without depletion (FcγRIIa binding efficiency). The FcγRIIa binding efficiency was significantly reduced after the NT specific antibodies were depleted (P=0.021).

FIG. 15 illustrates the results of vaccination with a combination of RTS,S and a CSP N-terminal PCMS antigen A) as previously, Rabbits were vaccinated with full-length CSP protein. After vaccination, serum was tested for IgG reactivity to full length CSP (FL-CSP), and the different regions of CSP—N-terminal (NT), C-terminal (CT), and NANP-repeat regions. Results demonstrate that vaccination with full length CSP does not effectively generate IgG to the NT region. B) Mice were vaccinated with a mixture of CSP corresponding to the RTS,S vaccine construct sequence either on its own (RTS,S alone), or mixed with a synthetic peptide sequence from the N-terminal region of CSP (NT+RTS,S). After vaccination, mouse serum was tested for the presence of IgG to the N-terminal (NT), C-terminal (CT), and NANP-repeat regions of CSP. Results demonstrate that a mixture of the RTS,S immunogen and the NT peptide immunogen effectively generates IgG to all 3 regions of CSP. Group 1: 5 μg RTS,S immunogen per dose. Group 2: 5 μg RTS,S immunogen+10 μg NT peptide. Vaccines were formulated with Quil-A adjuvant, and 3 doses were given

### DETAILED DESCRIPTION OF PARTICULAR EMBODIMENTS

Unless defined otherwise, all technical and scientific terms used herein have the same meaning as commonly understood by those of ordinary skill in the art to which the invention belongs.

Currently, the most advanced and most efficacious malaria vaccine is RTS,S and is based on the CSP antigen. However, the vaccine has only modest efficacy and new vaccines are needed that induced potent protective immunity. While this point is well recognized in the field, how to achieve better efficacy and induced more potent immune responses is unclear. The protective efficacy of the vaccine, and other vaccines based on CSP, is mediated by antibodies. One approach to achieve higher efficacy is to design vaccine immunogens that generate better and more potent functional antibodies (antibodies that contribute to rather than distract from vaccine efficiency). In accordance with the present description, the inventors have established that antibodies to CSP can function by interacting with complement protein in blood and with phagocytes (especially neutrophils). The RTS,S vaccine (and a related vaccine called R21) includes only the C-terminal region of CSP and part of the central repeat region. In accordance with the present description, the inventors have established that functional antibody activity is increased if antibodies target the N-terminal region of CSP, which is a region that is not part of the RTS,S vaccine. Epitopes have been identified in the N-terminal region that elicit functional antibodies allowing for the design of better vaccine constructs. Antibodies to the N-terminal region have been produced and these antibodies have good functional activity. Accordingly, in one embodiment it is proposed to make and/or use nanocarrier-based compositions comprising CSP antigens including N-terminal peptides as described herein, or the N-terminal peptides without peptides representing central or CT subregions of CSP antigen. Nanocarriers include VLPs including the HBV vectors such as those derived from human or hepadnovirus HBV vector, (see Kurtovic et al., 2021) or bacteriophage vectors.

By “derivative” or “modified” is meant a peptide or polypeptide or nucleic acid that has been derived from a basic or parental sequence by making changes thereto, for example by conjugation or complexing with other chemical moieties or by post-translational modification techniques as would be understood in the art. The terms also include parts or fragments of CSP where the sequence is substantially the same as the parental sequence as it relates to the part or fragment. The terms also include within their scope alterations that have been made to a parent sequence including a part or fragment thereof, including substitutions, additions, or deletions that provide for functionally equivalent or functionally improved molecules. Modified forms of CSP and its parts are known in the art and further proposed herein. Derivatives also include molecules having a percent amino acid or polynucleotide sequence identity over a window of comparison after optimal alignment. In one embodiment the percentage identity is at least 80%-99% including any number in between 80 and 99. Derivatives further include analogues, forms comprising heterologous elements such as spacers, native or non-native T-cell epitopes, and pro-drugs of the peptide and nanocarriers comprising the peptide.

Spacers may be oligo- or polypeptide molecules that link domains of an antigen and is flexible enough to allow antigen recognition and binding. A spacer domain may comprise or encode up to about 300 amino acids but is generally shorter.

By “isolated” is meant material that is substantially or essentially free from components that normally accompany it in its native state.

The term “subject,” includes patient, refer to any subject, particularly a human subject for whom prophylaxis or therapy is desired. The subject may be in need of prophylaxis or treatment, however, it will be understood that the aforementioned terms do not imply that symptoms of malaria or infection are present.

The terms “peptide”, “polypeptide”, “protein”, “glycoprotein” etc are interchangeable and do not imply any particular size.

The term “polynucleotide” or “nucleic acid” as used herein designates any form of nucleic acid known in the art such as mRNA, RNA, CRNA, cDNA or DNA or mixtures thereof. The term typically refers to oligonucleotides greater than 30 nucleotides in length.

The term sequence “identity” as used herein refers to the extent that sequences are identical on a nucleotide-by-nucleotide basis or an amino acid-by-amino acid basis over a window of comparison. Thus, a “percentage of sequence identity” is calculated by comparing two optimally aligned sequences over the window of comparison, determining the number of positions at which the identical nucleic acid base {e.g., A, T, C, G, U) or the identical amino acid residue (e.g., Ala, Pro, Ser, Thr, Gly, Val, Leu, lie, Phe, Tyr, Trp, Lys, Arg, His, Asp, Glu, Asn, Gln, Cys and Met) occurs in both sequences to yield the number of matched positions, dividing the number of matched positions by the total number of positions in the window of comparison (i.e., the window size), and multiplying the result by 100 to yield the percentage of sequence identity. For the purposes of the present invention, “sequence identity” may be understood to mean the “match percentage” calculated by the DNASIS computer program (Version 2.5 for Windows; available from Hitachi Software Engineering Co., Ltd., South San Francisco, California, USA) using standard defaults as used in the reference manual accompanying the software. Amino acid sequence identity may also be determined using the EMBOSS Pairwise Alignment Algorithms tool available from The European Bioinformatics Institute (EMBL-EBI), which is part of the European Molecular Biology Laboratory. This tool is accessible at the website located at www.ebi.ac.uk/Tools/emboss/align/. This tool utilizes the Needleman-Wunsch global alignment algorithm (Needleman and Wunsch, 1970). Default settings are utilized which include Gap Open: 10.0 and Gap Extend 0.5. The default matrix “Blosum62” is utilized for amino acid sequences and the default matrix.

The term sequence “similarity” refers to the percentage number of amino acids that are identical or constitute conservative amino acid substitutions as defined in Table 1 below. Similarity may be determined using sequence comparison programs such as GAP (Deveraux et al, 1984 Nucleic Acids Research 12: 387-395). In this way, sequences of a similar or substantially different length to those cited herein might be compared by insertion of gaps into the alignment, such gaps being determined, for example, by the comparison algorithm used by GAP.

APCMS peptide may be a peptide with 75, 76, 77, 78, 79, 80, 81, 82, 83, 84, 85, 86, 87, 88, 89, 90, 91, 92, 93, 94, 95, 96, 97, 98, 99, or 100% identity (or any derivable range therein) to a peptide of the disclosure. The peptide or polypeptide may have one or more conservative or non-conservative substitutions. Substitutional variants typically contain the exchange of one amino acid for another at one or more sites within the protein, and may be designed to modulate one or more properties of the polypeptide, with or without the loss of other functions or properties. Substitutions may be conservative, that is, one amino acid is replaced with one of similar shape and charge. Conservative substitutions are well known in the art and include, for example, the changes of: alanine to serine; arginine to lysine; asparagine to glutamine or histidine; aspartate to glutamate; cysteine to serine; glutamine to asparagine; glutamate to aspartate; glycine to proline; histidine to asparagine or glutamine; isoleucine to leucine or valine; leucine to valine or isoleucine; lysine to arginine; methionine to leucine or isoleucine; phenylalanine to tyrosine, leucine or methionine; serine to threonine; threonine to serine; tryptophan to tyrosine; tyrosine to tryptophan or phenylalanine; and valine to isoleucine or leucine. Alternatively, substitutions may be non-conservative such that a function or activity of the polypeptide is affected. Non-conservative changes typically involve substituting a residue with one that is chemically dissimilar, such as a polar or charged amino acid for a nonpolar or uncharged amino acid, and vice versa. Indicative substitutions are listed in Tables 2 and 3.

The PCMS antigens or peptides described herein may include 1, 2, 3, 4, 5, 6, 7, 8, 9, 10, 11, 12, 13, 14, 15, or more (or any derivable range therein) variant amino acids within at least, or at most 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, or 104 contiguous amino acids, or any range derivable therein, of a peptide of the disclosure.

A polypeptide segment as described herein may include 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104 or more contiguous amino acids, or any range derivable therein of a peptide of the disclosure.

The PCMS peptides described herein may be of a fixed length of at least, at most, or exactly 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110 or more amino acids (or any derivable range therein).

### Immune Cell Types

Antibodies to sporozoites antigens are typically dominated by the IgG1 and IgG3 subclasses (Irani, V., et al. Molecular properties of human IgG subclasses and their implications for designing therapeutic monoclonal antibodies against infectious diseases. Molecular Immunology (2015). These subclasses have strong potential to interact with FcγRs suggesting that FcγR-mediated mechanisms may also be important against sporozoites. IgG1 and IgG3 also have a strong potential to fix and activate complement. Currently, there is little known about these potential mechanisms in immunity to Plasmodium sporozoites.

The major phagocytic cells types are monocytes and neutrophils, which account for 2-10% and 45%-70% of peripheral white blood cells, respectively. Both monocytes and neutrophils also have wider immune impacts through expression of activation markers, cytokines, and chemokines (reviewed in Gordon, S. Phagocytosis: An Immunobiologic Process. Immunity 44, 463-475 (2016)). Natural killer (NK) cells are also active against opsonised pathogens through interactions with FcγRIIIa expressed on their surface.

In addition to phagocytosis, neutrophils and natural killer (NK) cells can also kill cells through antibody-dependent cellular cytotoxicity (ADCC) (Kolaczkowska, E. & Kubes, P. Neutrophil recruitment and function in health and inflammation. Nat Rev Immunol 13, 159-175 (2013)).

FcγR expression patterns differ between monocytes and neutrophils, which impacts on their functions in immunity.

Since sporozoites represent the first step in initiating malaria infection, sporozoites would typically encounter potential phagocytic cells in a resting, or homoeostatic, state rather than an activated state. Resting-stage monocytes predominantly express FcγRI and FcγRIIa, with a small subset expressing FcγRIIIa. In contrast, resting-stage neutrophils express FcγRIIa and FcγRIIIb, and low levels of FcγRIIIa.

IgG subclass, glycosylation, and epitope specificity can each impact on interactions with different FcγRs, and ability to fix and activate complement.

Knowledge of specific regions and epitopes of CSP that are targeted by potent functional antibodies could open new avenue for developing more efficacious vaccines.

In the work leading up to the present application, the ability of antibodies to interact with Fcγ-receptors (FcγR) on various cell types was investigated and their ability to promote opsonic phagocytosis of malaria parasites or direct parasite cytotoxicity.

It was proposed that defining FcγR-mediated mechanisms targeting sporozoites, and the major vaccine antigen CSP, would reveal novel insights that could be exploited to enhance the protective efficacy of sporozoite-based vaccines. The results described and enabled herein reveal an important role for specific FcγRs and neutrophils in mediating antibody-dependent opsonic phagocytosis of sporozoites via specific N-terminal epitopes. Antibodies to the N-terminal epitopes can also promote complement fixation, which may further contribute to killing of sporozoites.

The present application enables malaria vaccines or immunogenic compositions comprising CSP N-terminal epitopes, antibodies thereto and methods of screening for antibodies that mediate phagocytosis/sporozoite cell death.

The present application also provides kits or surfaces comprising the PCMS peptides described herein. The present application contemplates PCMS peptides in lyophilised or dried or in solution, gel or in matrix formats.

The present disclosure is predicated, in part, on the discovery that there is a higher concentration of antibody epitopes for phagocytosis contained within the NT region of CSP relative to full length CSP, CT and NANP subregions, and that antibodies against these epitopes are functionally active in vivo. This discovery allows for a different approach for screening vaccine candidates and has identified epitopes of interest that provide a range of DNA, polypeptide, cell or particle based vaccines comprising and/or encoding these epitopes.

Accordingly, in one aspect the disclosure provides an immunogenic composition comprising or encoding a modified PfCSP or a fragment thereof comprising N-terminal epitopes that stimulate an antibody response that stimulates sporozoite opsonisation and immune cell mediated clearance in a subject.

Recombinant CSP proteins were generated based on the 3D7 allele P. falciparum CSP (XP_001351122), expressed in the mammalian HEK293F cell line. CSP contains 397 amino acids and can be divided into several domains: The N-terminal signal peptide (amino acids 1-18), the N-terminal domain (amino acids 19-104) that contains a free cysteine residue at position 25, the central repeat region consisting of 4 NVDP and 38 NANP repeats (amino acids 105-272), and the C-terminal domain (amino acids 273-398) that contains a predicted GPI anchor Omega site at Cys-374.

Derivatives also include molecules having a percent amino acid or polynucleotide sequence identity over a window of comparison after optimal alignment. In one embodiment the percentage identity is at least 80%-99% including any number in between 80 and 99 as discussed further herein.

Suitable assays for the biological activity of peptides or epitopes or constructs or sporozoites comprising them are known to the skilled addressee and are described in the examples and Figures.

VLP technologies are known in the art for presenting antigens to the immune system. Antigens may be administered in the form of compositions comprising VLP presenting the antigen including HBV vectors such as human or duck HBV vectors.

In some embodiments, alternative or additional markers of peptide or construct activity or ability to induce an effective phagocytic/cytotoxic effect include: parasite cell death, neutrophil activity, antibody specificity assays etc.

A nucleic acid encoding all or part of a PCMS antigen or peptide may contain a contiguous nucleic acid sequence of: 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 441, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, 1000, 1010, 1020, 1030, 1040, 1050, 1060, 1070, 1080, 1090, 1095, 1100, 1500, 2000, 2500, 3000, 3500, 4000, 4500, 5000, 5500, 6000, 6500, 7000, 7500, 8000, 9000, 10000, or more nucleotides, nucleosides, or base pairs, including all values and ranges there between, of a polynucleotide encoding one or more amino acid sequence described or referenced herein. It also is contemplated that a particular polypeptide may be encoded by nucleic acids containing variations having slightly different nucleic acid sequences but, nonetheless, encode the same or substantially similar protein. Codon optimisation strategies are known in the art.

In particular embodiments, the application provides isolated nucleic acid segments and vectors incorporating nucleic acid sequences that encode a peptide of the disclosure. The term “recombinant” may be used in conjunction with a polynucleotide or polypeptide and generally refers to a polypeptide or polynucleotide produced and/or manipulated in vitro or that is a replication product of such a molecule.

The nucleic acid segments used in the current disclosure can be combined with other nucleic acid sequences, such as promoters, polyadenylation signals, additional restriction enzyme sites, multiple cloning sites, other coding segments, and the like, such that their overall length may vary considerably. It is therefore contemplated that a nucleic acid fragment of almost any length may be employed, with the total length preferably being limited by the ease of preparation and use in the intended recombinant nucleic acid protocol. In some cases, a nucleic acid sequence may encode a polypeptide sequence with additional heterologous coding sequences, for example to allow for purification of the polypeptide, transport, secretion, post-translational modification, or for therapeutic benefits such as targeting or efficacy. As discussed above, a tag or other heterologous polypeptide may be added to the modified polypeptide-encoding sequence, wherein “heterologous” refers to a polypeptide that is not the same as the modified polypeptide.

In certain embodiments, the current disclosure provides polynucleotide variants having substantial identity to the sequences disclosed herein; those comprising at least 70%, 75%, 80%, 85%, 90%, 95%, 96%, 97%, 98%, or 99% or higher sequence identity, including all values and ranges there between, compared to a polynucleotide sequence of this disclosure using the methods described herein (e.g., BLAST analysis using standard parameters).

The disclosure also contemplates the use of polynucleotides which are complementary to all the above described polynucleotides. Polynucleotides may be codon optimized for expression in hosts/subjects.

A construct or vector for expressing a PCM antigen as described herein in a recipient cell can comprise one or more DNA regions comprising a promoter operably linked to a nucleotide sequence encoding the peptide. The promoter can be inducible or constitutive. Examples of suitable constitutive promoters include, e.g., an immediate early cytomegalovirus (CMV) promoter, an Elongation Growth Factor-1a (EF-1a) gene promoter, a simian virus 40 (SV40) early promoter, a mouse mammary tumor virus (MMTV) promoter, a human immunodeficiency virus (HIV) long terminal repeat (LTR) promoter, a MoMuLV promoter, an avian leukemia virus promoter, an Epstein-Barr virus immediate early promoter, a Rous sarcoma virus promoter, as well as human gene promoters such as, but not limited to, the actin promoter, the myosin promoter, the hemoglobin promoter, and the creatine kinase promoter. Examples of inducible promoters include, but are not limited to a metallothionine promoter, a glucocorticoid promoter, a progesterone promoter, and a tetracycline promoter.

The expression constructs may be generated by any suitable method including recombinant or synthetic techniques, utilizing a range of vectors known and available in the art such as plasmids, bacteriophage, baculovirus, mammalian virus, artificial chromosomes, among others. The expression constructs can be circular or linear, and should be suitable for replication and integration into eukaryotes. Viruses, which are useful as vectors include, but are not limited to, retroviruses, adenoviruses, adeno-associated viruses, herpes viruses and lentiviruses. A number of viral based systems have been developed for gene transfer into mammalian cells. For example, retroviruses provide a convenient platform for gene delivery systems. A selected gene can be inserted into a vector and packaged in retroviral particles using techniques known in the art. The recombinant virus can then be isolated and delivered to the subject stem cells. A number of retroviral systems are known in the art.

In a specific embodiments where the immunogenic composition is provided as a nucleic acid encoding the peptide, the nucleic acid may be administered in vivo to promote expression of its encoded protein, by constructing it as part of an appropriate nucleic acid expression vector and administering it so that it becomes intracellular (e.g., by use of a retroviral vector, by direct injection, by use of microparticle bombardment, by coating with lipids or cell-surface receptors or transfecting agents, or by administering it in linkage to a homeobox-like peptide or other intracellular targeting moiety.

A nucleic acid molecule as described herein may in any form such as DNA or RNA, including in vitro transcribed RNA or synthetic RNA. Nucleic acids include genomic DNA, cDNA, RNA, recombinantly produced and chemically synthesized molecules and modified forms thereof. A nucleic acid molecule may be single stranded or double stranded and linear or closed covalently to form a circle. The RNA may be modified by stabilizing sequences, capping, and polyadenylation. RNA or DNA and may be delivered as plasmids to express the peptide. RNA-based approaches are routinely available.

The term “RNA” relates to a molecule which comprises ribonucleotide residues and preferably being entirely or substantially composed of ribonucleotide residues. “Ribonucleotide” relates to a nucleotide with a hydroxyl group at the 2′-position of a β-D-ribofuranosyl group. The term includes double stranded RNA, single stranded RNA, isolated RNA such as partially purified RNA, essentially pure RNA, synthetic RNA, recombinantly produced RNA, as well as modified RNA that differs from naturally occurring RNA by the addition, deletion, substitution and/or alteration of one or more nucleotides. Such alterations can include addition of non-nucleotide material, such as to the end(s) of a RNA or internally, for example at one or more nucleotides of the RNA. Nucleotides in RNA molecules can also comprise non-standard nucleotides, such as non-naturally occurring nucleotides or chemically synthesized nucleotides or deoxynucleotides. These altered RNAs can be referred to as analogs or analogs of naturally-occurring RNA.

An optimised mRNA based composition could comprise a 5′ and 3′ non translated region (5′-UTR, 3′-UTR) that optimises translation efficiency and intracellular stability as known in the art. An open reading frame encoding the RHIM-interating peptide of Structure 1. In one embodiment, removal of uncapped 5 ‘-triphosphates can be achieved by treating RNA with a phosphatase. RNA may have modified ribonucleotides in order to increase its stability and/or decrease cytotoxicity. For example, in one embodiment, in the RNA, 5-methylcytidine is substituted partially or completely, for cytidine. In one embodiment, the term “modification” relates to providing an RNA with a 5’-cap or 5′-cap analog. The term “5′-cap” refers to a cap structure found on the 5′-end of an mRNA molecule and generally consists of a guanosine nucleotide connected to the mRNA via an unusual 5′ to 5′ triphosphate linkage. In one embodiment, this guanosine is methylated at the 7-position. The term “conventional 5′-cap” refers to a naturally occurring RNA 5′-cap, preferably to the 7-methylguanosine cap. The term “5′-cap” includes a 5′-cap analog that resembles the RNA cap structure and is modified to possess the ability to stabilize RNA and/or enhance translation of RNA. Providing an RNA with a 5′-cap or 5′-cap analog may be achieved by in vitro transcription of a DNA template in the presence of said 5′-cap or 5′-cap analog, wherein said 5′-cap is co-transcriptionally incorporated into the generated RNA strand, or the RNA may be generated, for example, by in vitro transcription, and the 5′-cap may be attached to the RNA post-transcriptionally using capping enzymes, for example, capping enzymes of vaccinia virus.

A further modification of RNA may be an extension or truncation of the naturally occurring poly(A) tail or an alteration of the 5′- or 3 ‘-untranslated regions (UTR) such as introduction of a UTR which is not related to the coding region of said RNA, for example, the exchange of the existing 3’-UTR with or the insertion of one or more, preferably two copies of a 3′-UTR derived from a globin gene, such as alpha2-globin, alpha1-globin, beta-globin. RNA having an unmasked poly-A sequence is translated more efficiently than RNA having a masked poly-A sequence. In order to increase stability and/or expression of the RNA it may be modified so as to be present in conjunction with a poly-A sequence, preferably having a length of 10 to 500, more preferably 30 to 300, even more preferably 65 to 200 and especially 100 to 150 adenosine residues. In order to increase expression of the RNA it may be modified within the coding region so as to increase the GC-content to increase mRNA stability and to perform a codon optimization and, thus, enhance translation in cells. Modified mRNA may be synthesised enzymatically and packaged into nanoparticles such as lipid nanoparticles and administered, for example intramuscularly.

The nucleic acid molecule can be entrapped in microcapsules prepared, for example, by coacervation techniques or by interfacial polymerization, in colloidal drug delivery systems (e.g., liposomes, microspheres, microemulsions, nanoparticles and nanocapsules), or in macroemulsions. Such techniques are known in the art and broadly disclosed in Remington, the Science and Practice of Pharmacy, 20th Edition, Remington, J., ed. (2000) and updates thereof.

The present application extends to host cells containing the subject peptides or their encoding nucleic acid molecules. As used herein, the terms “cell,” “cell line,” and “cell culture” may be used interchangeably. All of these terms also include their progeny, which is any and all subsequent generations. It is understood that all progeny may not be identical due to deliberate or inadvertent mutations. In the context of expressing a heterologous nucleic acid sequence, “host cell” refers to a prokaryotic or eukaryotic cell, and it includes any transformable organism that is capable of replicating a vector or expressing a heterologous gene encoded by a vector. A host cell can, and has been, used as a recipient for vectors or viruses. A host cell may be “transfected” or “transformed,” which refers to a process by which exogenous nucleic acid, such as a recombinant protein-encoding sequence, is transferred or introduced into the host cell. A transformed cell includes the primary subject cell and its progeny.

Host cells may be derived from prokaryotes or eukaryotes, including bacteria, yeast cells, insect cells, and mammalian cells for replication of the vector or expression of part or all of the nucleic acid sequence(s). Numerous cell lines and cultures are available for use as a host cell, and they can be obtained through the American Type Culture Collection (ATCC), which is an organization that serves as an archive for living cultures and genetic materials (www.atcc.org).

Numerous expression systems exist that comprise at least a part or all of the compositions discussed above. Prokaryote- and/or eukaryote-based systems can be employed for use with the present invention to produce nucleic acid sequences, or their cognate peptides. Many such systems are commercially and widely available.

The insect cell/baculovirus system can produce a high level of protein expression of a heterologous nucleic acid segment, such as described in U.S. Pat. Nos. 5,871,986, 4,879,236, both herein incorporated by reference, and which can be bought, for example, under the name Bac-to-Bac® Baculovirus Expression System from ThermoFisher and BacPAK™ Baculovirus Expression System from Takara®.

In addition, other examples of expression systems include STRATAGENE®'s COMPLETE CONTROL inducible Mammalian Expression System, which involves a synthetic ecdysone-inducible receptor, or its pET Expression System, an E. coli expression system. Another example of an inducible expression system is available from ThermoFisher®, which carries the T-REX™ (tetracycline-regulated expression) System, an inducible mammalian expression system that uses the full-length CMV promoter. ThermoFisher® also provides a yeast expression system designed for high-level production of recombinant proteins in the yeast genus Pichia. One of skill in the art would know how to express a vector, such as an expression construct, to produce a nucleic acid sequence or its cognate peptide.

### Treatment and Administration

In accordance with this disclosure the subject composition or pharmaceutical composition may be administered or a pharmaceutically acceptable salt, hydrate, tautomer, sterioisomer, pro-drug thereof.

For combination therapies, each component of the combination therapy may be administered at the same time, or sequentially in any order, or at different times, so as to provide the desired effect. Alternatively, the components may be together in a single dosage unit as a combination product. When administered separately, it may be preferred for the components to be administered by the same or different routes of administration. Administration may be by administering the antigen or a polynucleotide expressing the antigen and vaccination protocols may alternate between protein and nucleic acid.

The compositions may be delivered by injection, by topical or mucosal application, by inhalation or via oral route including modified release modes, over periods of time and in amounts which are effective to effect or optimise IgG subclass responses, preferably reduce IgM responses that may inhibit FcγR interactions. Administration may be systemic (e.g., parenteral via for example intravenous, intraperitoneal, intradermal, sub cutaneous or intramuscular routes) or targeted.

The amount of the peptide, composition, VLP or vaccine to be administered may be determined by standard clinical techniques by those of average skill within the art. In addition, in vitro assays may optionally be employed to help identify optimal dosage ranges. The precise dose to be employed will also depend on the nature of the agent and other clinical factors (such as the condition of the subject their weight, the route of administration and type of composition). The precise dosage to be therapeutically or prophylactically effective and non-detrimental can be determined by those skilled in the art. Pharmaceutical compositions are conveniently prepared according to conventional pharmaceutical compounding techniques. See, for example, Remington, the Science and Practice of Pharmacy, 20th Edition, Remington, J., ed. (2000) and later editions.

However, suitable dosage ranges for intravenous administration of the peptide of the present invention are generally about 1.25-5 micrograms of active compound per kilogram (Kg) body weight. Suitable dosage ranges for intranasal administration are generally about 0.01 pg/kg body weight to 1 mg/kg body weight. Effective doses may be extrapolated from dose-response curves derived from in vitro or animal model test systems. Suppositories generally contain active ingredient in the range of 0.5% to 10% by weight; oral compositions preferably contain 10% to 95% active ingredient.

In another embodiment, the present invention provides a method of eliciting a humoral or cell-mediated immune response in a subject or patient, the method comprising administering to the subject an effective amount of a composition comprising the herein disclosed antigen.

The term “treatment” refers to any measurable or statistically significant amelioration in at least some subjects in one or more symptoms of malaria or in the risk of developing advanced symptoms of malaria or the risk of transmitting Plasmodium sp.. The terms “prevention” and “prophylaxis” and the like are used interchangeably and include administration of a composition of the present invention to a subject not known to be infected with Plasmodium sp. for the purpose of prevention or attenuating a subsequent infection or reducing the risk of becoming infected or reducing the severity or onset of a condition or signs of a condition associated with Plasmodium infection. The administration of a vaccine composition is generally for prophylactic purposes. The prophylactic administration of the composition serves to prevent or attenuate any subsequent infection. A “pharmacologically acceptable” composition is one tolerated by a recipient patient. It is contemplated that an effective amount of the vaccine is administered. An “effective amount” is an amount sufficient to achieve a desired biological effect such as to induce enough humoral or cellular immunity. This may be dependent upon the type of vaccine, the age, sex, health, and weight of the recipient. Examples of desired biological effects include, but are not limited to, production of no symptoms, reduction in symptoms, reduction in parasite titre in tissues complete protection against infection, and partial protection. In some embodiments, a vaccine or composition of the present invention is physiologically significant if its presence results in a detectable change in the physiology of a recipient patient that enhances or indicates an enhancement in at least one primary or secondary humoral or cellular immune response against Plasmodium species or strains.

A “pharmaceutically acceptable carrier and or a diluent” is a pharmaceutical vehicle comprised of a material that is not otherwise undesirable i.e., it is unlikely to cause a substantial adverse reaction by itself or with the active composition. Carriers may include all solvents, dispersion media, coatings, antibacterial and antifungal agents, agents for adjusting tonicity, increasing or decreasing absorption or clearance rates, buffers for maintaining pH, chelating agents, membrane or barrier crossing agents. A pharmaceutically acceptable salt is a salt that is not otherwise undesirable. The agent or composition comprising the agent may be administered in the form of pharmaceutically acceptable non-toxic salts, such as acid addition salts or metal complexes.

For oral administration, the compositions can be formulated into solid or liquid preparations such as capsules, pills, tablets, lozenges, powders, suspensions or emulsions. In preparing the compositions in oral dosage form, any of the usual pharmaceutical media may be employed, such as, for example, water, glycols, oils, alcohols, flavoring agents, preservatives, coloring agents, suspending agents, and the like in the case of oral liquid preparations (such as, for example, suspensions, elixirs and solutions); or carriers such as starches, sugars, diluents, granulating agents, lubricants, binders, disintegrating agents and the like in the case of oral solid preparations (such as, for example, powders, capsules and tablets). Because of their ease in administration, tablets and capsules represent the most advantageous oral dosage unit form, in which case solid pharmaceutical carriers are obviously employed. Tablets may contain a binder such as tragacanth, corn starch or gelatin; a disintegrating agent, such as alginic acid; and a lubricant, such as magnesium stearate. If desired, tablets may be sugar-coated or enteric-coated by standard techniques. The active composition can be encapsulated to make it stable to passage through the gastrointestinal tract. See for example, International Patent Publication No. WO 96/11698.

For parenteral administration, the composition may be dissolved in a carrier and administered as a solution or a suspension. For transmucosal or transdermal (including patch) delivery, appropriate penetrants known in the art are used for delivering the composition. For inhalation, delivery uses any convenient system such as dry powder aerosol, liquid delivery systems, air jet nebulizers, propellant systems. For example, the formulation can be administered in the form of an aerosol or mist. The compositions may also be delivered in a sustained delivery or sustained release format. For example, biodegradable microspheres or capsules or other polymer configurations capable of sustained delivery can be included in the formulation. Formulations can be modified to alter pharmacokinetics and biodistribution. For a general discussion of pharmacokinetics, see, e.g., Remington's Pharmaceutical Sciences, 1990 (supra). In some embodiments the formulations may be incorporated in lipid monolayers or bilayers such as liposomes or micelles. Targeting therapies known in the art may be used to deliver the agents more specifically to certain types of cells or tissues.

Antibodies generated against the PCMS NT CSP peptides disclosed herein are also contemplated, such as monoclonal antibodies, or derivatives or analogs thereof, include without limitation: Fv fragments; single chain Fv (scFv) fragments; Fab′ fragments; F(ab′)2 fragments; humanized antibodies and antibody fragments; camelized antibodies and antibody fragments, and multivalent versions of the foregoing. Multivalent binding reagents also may be used, as appropriate, including without limitation: monospecific or bispecific antibodies; such as disulfide stabilized Fv fragments, scFv tandems (scFv) fragments, diabodies, tribodies or tetrabodies, which typically are covalently linked or otherwise stabilized (i.e. leucine zipper or helix stabilized) scFv fragments.

The term “antibody fragments”, as used herein, include any portion of an antibody that retains the ability to bind to the epitope recognized by the full length antibody. Examples of antibody fragments include, but are not limited to, Fab, Fab′ and F(ab′)₂, Fd, single-chain Fvs (scFv), disulfide-linked Fvs (dsFv), and fragments comprising either a V_{L} or V_{H} region. Antigen-binding fragments of antibodies can comprise the variable region(s) alone or in combination with a portion of the hinge region, CH1, CH2, CH3, or a combination thereof. Preferably, the antibody fragments contain all six CDRs of the whole antibody, although fragments containing fewer than all six CDRs may also be functional.

“Single-chain FVs” (“scFvs”) are antigen-binding fragments that contain the heavy chain variable region (V_{H}) of an antibody linked to the light chain variable region (V_{L}) of the antibody in a single polypeptide, but lack some or all of the constant domains of the antibody. The linkage between the V_{H} and V_{L} can be achieved through a short, flexible peptide selected to assure that the proper three-dimensional folding of the V_{L} and V_{H} regions occurs to maintain the target molecule binding-specificity of the whole antibody from which the scFv is derived. scFvs lack some or all of the constant domains of antibodies.

Methods of making antigen or epitope-specific binding agents, including antibodies and their derivatives and analogs and aptamers, are well-known in the art. Polyclonal antibodies can be generated by immunization of an animal. Monoclonal antibodies, derivatives and analogs can be prepared according to standard methodology.

Methods involving conventional molecular biology techniques are described herein. Such techniques are generally known in the art and are described in detail in methodology treatises such as Molecular Cloning: A Laboratory Manual, 3rd ed., vol. 1-3, ed. Sambrook et al., Cold Spring Harbor Laboratory Press, Cold Spring Harbor, N.Y., (2001); and Current Protocols in Molecular Biology, ed. Ausubel et al., Greene Publishing and Wiley-Interscience, New York, (1992) (with periodic updates). Immunology techniques are generally known in the art and are described in detail in methodology treatises such as Current Protocols in Immunology, ed. Coligan et al., Greene Publishing and Wiley-Interscience, New York, (1992) (with periodic updates); Advances in Immunology, volume 93, ed. Frederick W. Alt, Academic Press, Burlington, Mass., (2007); Making and Using Antibodies: A Practical Handbook, eds. Gary C. Howard and Matthew R. Kaser, CRC Press, Boca Raton, Fl, (2006); Medical Immunology, 6th ed., edited by Gabriel Virella, Informa Healthcare Press, London, England, (2007); and Harlow and Lane ANTIBODIES: A Laboratory Manual, Second edition Cold Spring Harbor Laboratory Press, Cold Spring Harbor, N.Y., (2014). Conventional methods of gene transfer and gene therapy may also be adapted for use in the present invention. See, e.g., Gene Therapy: Principles and Applications, ed. T. Blankenstein, Springer Verlag, 1999; Gene Therapy Protocols (Methods in Molecular Medicine), ed. P. D. Robbins, Humana Press, 1997; Viral Vectors for Gene Therapy: Methods and Protocols, ed. Otto-Wilhelm Merten and Mohammed Al-Rubeai, Humana Press, 2011; and Nonviral Vectors for Gene Therapy: Methods and Protocols, ed. Mark A. Findeis, Humana Press, 2010. Amino Acids 2018 January; 50(1):39-68. doi: 10.1007/s00726-017-2516-0. Epub 2017 Nov. 28. Peptides may be produced by any method known in the art. Peptides and their encoding nucleic acids may be modified by many different strategies known in the art to modify their stability, binding, activity or detectability, expression, ability to penetrate cells etc. Peptides and their nucleic acids may be produced synthetically and may be obtained through the application of well known recombinant nucleic acid techniques. Peptides may be synthesised using conventional liquid or increasingly solid phase synthesis techniques. For example, initial reference may be made to solution synthesis or solid phase synthesis as described, for example, by Atherton and Sheppard in SOLID PHASE PEPTIDE SYNTHESIS: A PRACTICAL APPROACH (IRL Press at Oxford University, Oxford, England, 1989), see particularly Chapter 9, or by Roberge et al. (1995 Science 269: 202).

A key role has been identified herein for neutrophils in clearance of sporozoites in blood, mediated by FcγRIIa and FcγRIII interactions with IgG1 and IgG3 targeting the major sporozoite antigen, CSP and particularly the N-terminal region defined herein. The present application describes the critical role of FcγRIIa and FcγRIII in this immune mechanism, and particularly the importance of FcγRIII. Antibodies to CSP and particularly the N-terminal epitopes described herein promote NK cell activity, which is mediated via FcγRIIIa.

Using intact sporozoites and antigen-coated beads it has been established that phagocytosis, in whole blood, is predominantly mediated by neutrophils with a low-level activity observed with monocytes.

Key mechanisms have been identified involving phagocytosis by assessing the impact of blocking FcγRs on cell surfaces and through using novel FcγR-dimers as probes to bind antigen-antibody complexes.

The CSP is a major target of these functional antibodies and it is shown that all three regions of CSP can be targeted by antibodies to engage FcγR and promote opsonic phagocytosis. In particular, antibodies to the NT region had the greatest activity across a range of different assays, providing new vaccine constructs as described herein.

Further, it is disclosed herein that naturally acquired antibodies from individuals residing a malaria-endemic region were capable of promoting opsonic phagocytosis of sporozoites. The acquisition of functional antibodies was age-dependent and higher levels of opsonic phagocytosis activity and binding of FcγRIIa and FcγRIII were only observed with samples from adults but not young children. Functional activity correlated with IgG1 and IgG3 subclasses.

After inoculation into the skin, sporozoites travel through the dermis, and circulate in the blood-stream before reaching liver sinusoids and achieving invasion of hepatocytes. During this process, which can take several hours, there is substantial exposure to neutrophils, the most abundant phagocyte in the blood, monocytes, and NK cells. The demonstration here that neutrophils in the peripheral blood effectively phagocytose antibody-opsonised sporozoites in vitro indicates this is a mechanism that occurs in vivo. Sporozoites are also exposed to dermal macrophages and liver Kupffer cells, which involve additional mechanisms of FcγR-mediated clearance.

The higher phagocytosis activity of neutrophils, but very low-level activity of monocytes, may relate to the presence and use of different FcγRs; it should be noted that neutrophils had a greater rate of phagocytosis, which was not simply explained by the higher abundance of neutrophils compared to monocytes. Opsonic phagocytosis is initiated by cross-linking FcγRs on the surface of phagocytes, and neutrophils and monocytes express different FcγRs on their surface. In our studies, we focused on resting stage monocytes and neutrophils as the sporozoite triggers minimal immune activation during the infection process. Resting stage monocytes express FcγRI and FcγRIIa with only small subsets of monocytes also expressing FcγRIIIa. On the other hand, resting neutrophils express FcγRIIa, FcγRIIIa and FcγRIIIb (Golay et al. 2019). Interestingly, blocking either FcγRIIa or FcγRIIIa/b inhibited phagocytosis activity by neutrophils, suggesting that both receptors are required for phagocytosis, which was supported by achieving great inhibition with blocking antibodies to FcγRIIa and FcγRIII used in combination. Blocking FcγRI had no effect on phagocytosis by neutrophils. Several observations suggested the higher importance of FcγRIII; there was generally greater inhibition of sporozoite phagocytosis by FcγRIII blockade, and FcγRIII binding by antibodies was more highly correlated with phagocytosis than FcγRIIa in multivariate analysis.

FcγRIIIb, which is GPI-anchored, is the most abundant FcγRIII type expressed on neutrophils, but neutrophils also express FcγRIIIa, which has a transmembrane and cytoplasmic domain for intracellular signalling. For phagocytosis, intracellular signalling may occur via FcγRIIIa or FcγRIIa, with binding to FcγRIIIb also being important, as indicated by our findings. With THP-1 cells, phagocytosis was effectively inhibited by blockade of FcγRI, but minimal inhibition was seen with blocking FcγRIIa or FcγRIII. This suggests that phagocytosis by THP-1 cells is mainly mediated by FcγRI. If FcγRIII and FcγRIIa act cooperatively in phagocytosis, this may explain why there was limited activity of FcγRIIa in phagocytosis by monocytes. NK cells express FcγRIIIa, and we established that antibodies to CSP can promote NK activation through interaction with FcγRIIIa.

Existing studies on antibody-mediated opsonic phagocytosis in malaria have largely used the THP-1 cell line as a model, using standard conditions with FCS (but not including human serum). A recent study reported that opsonic phagocytosis by THP-1 cells was not correlated with protection in a RTS,S phase I/IIa trial in malaria naïve adults. The present application shows that phagocytosis by THP-1 cells is predominantly mediated through FcγRI, highlighting differences in functional mechanisms compared to neutrophils. Additionally, non-specific IgG in human serum can also reduce THP-1 cell phagocytosis activity because FcγRI interactions are inhibited by monomeric IgG presented in human plasma. Therefore, using THP-1 cells does not appear to be a good approach for studying the opsonic phagocytosis against sporozoites.

CSP is the most abundant antigen of sporozoites and the leading vaccine antigen. The present application shows that all three regions of CSP (NT, CT, and central repeat regions) can be targeted by antibodies that engage FcγRIIa and FcγRIII to promote phagocytosis of sporozoites by neutrophils, thereby ascribing new functions for antibodies to these regions that are likely to contribute to immunity. In particular, the FcγR-mediated functional potential of antibodies to the NT region described herein was higher than other regions, as seen in phagocytosis assays using human antibodies with antigens coated onto beads, phagocytosis of sporozoites by rabbit antibodies raised to different regions, and FcγR-binding by human antibodies to different CSP regions. These findings suggest a greater focus on the NT region in vaccine design will be valuable; neither RTS,S or R21 vaccines that are based on CSP include the NT region; both vaccines contain only the central repeat (NANP repeats) and CT regions of CSP. A recent study showed antibodies to both the central repeat region and the CT region were associated with vaccine efficacy in the RTS,S phase III trial involving infants and young children.

The findings here that antibodies are naturally-acquired through exposure to malaria that promote FcγR-mediated phagocytosis by neutrophils further show that this is a mechanism that occurs in vivo. In a setting of high malaria transmission, it is shown that these functional antibodies are very low among young children who are most at risk of infection and severe malaria, but are present at much higher levels among some adults with lifelong exposure. Notably, functional antibodies to CSP were acquired much more slowly, and required greater exposure to develop, compared to antibodies that promote the phagocytosis of blood-stage merozoites. Using FcγR dimers as probes, the present application demonstrated that acquired antibodies to CSP can promote interactions with FcγRIIa and FcγRIII. The relative levels of activity with each receptor varied significantly among individuals, which is likely to impact on phagocytosis activity. Variations in specific FcγR binding will be influenced by IgG subclass profiles, epitopes targeted by antibodies, and IgG glycosylation (Irani, V., et al. Molecular properties of human IgG subclasses and their implications for designing therapeutic monoclonal antibodies against infectious diseases. Molecular immunology (2015)). IgG1 and IgG3, which have the highest FcγR-binding activity, predominated among acquired IgG subclasses and correlated with FcγR-binding activity.

Prior studies have also demonstrated that antibodies to sporozoites can inhibit sporozoite motility, cell traversal, and hepatocyte invasion as potential mechanisms mediating immunity. However, there is currently no consistent evidence showing these mechanisms correlate with protection against Plasmodium infection. In passive immunization models in animals, high levels of IgG to CSP can protect against experimental infection. However, similar high levels of protection, sustained over time, have not yet been achieved by vaccine-induced antibodies in clinical trials.

RTS,S induces very high CSP IgG levels, but only provides modest protection, suggesting that new strategies are needed to generate antibodies that have higher functional protective activity.

The present application harnessing FcγR-mediated mechanisms to generate more potent immunity by vaccines for higher levels of protection.

The present application describes a platform screen to study opsonic phagocytosis activity of antibodies to CSP, the key immune target of P. falciparum sporozoites. This platform enables screening for the presently disclosed functional antibody responses.

Future studies investigating the induction of these functional antibodies and their correlation with protection are warranted in phase II and phase III trials of RTS,S, and other CSP-based vaccines. We have conducted an initial study in a phase I/IIa trial of RTS,S vaccination followed by experimental infection challenge in healthy malaria-naïve adults (Kester, K. E., et al. Randomized, double-blind, phase 2a trial of falciparum malaria vaccines RTS,S/AS01B and RTS,S/AS02A in malaria-naive adults: safety, efficacy, and immunologic associates of protection. J Infect Dis 200, 337-346 (2009)). This initial study established that RTS,S vaccination does induce antibodies to CSP that promote binding of FcγRIIa and FcγRIII, as well as phagocytosis by neutrophils and these functional activities were associated with protection from infection, thus providing a useful baseline.

The specification discloses the importance of FcγR-mediated mechanisms in clearance of sporozoites from blood. This disclosure shows a key role for neutrophils in the phagocytic clearance of sporozoites, defined important roles for FcγRIIa and FcγRIII and established CSP as a key target for this functional activity. Monocytes also contribute to phagocytosis, but have lower activity, and antibodies can also promote NK cell ADCC activity. The specification establishes the higher functional activity of antibodies to the NT region of CSP, which was not included in the RTS,S vaccine. Further, new functional activities of antibodies to the CT region are described which have been associated with protection in RTS,S vaccine trials (Dobano, C., et al. Concentration and avidity of antibodies to different circumsporozoite epitopes correlate with RTS,S/AS01E malaria vaccine efficacy. Nat Commun 10, 2174 (2019)). Understanding and defining mechanisms that mediate immunity is crucial to enable the development of more efficacious vaccines. Harnessing this immune mechanism and novel targets in vaccine development provides a strategy for generating more potent immune response that provide a higher levels of sporozoite destruction and consequent protection against malaria.

The following embodiments are encompassed:

In one embodiment, CSP N-terminal (NT) peptides are PCMS antigens or epitopes that promote in a subject antibody dependent Phagocytosis or Complement Mediated killing of Sporozoites (PCMS). In particular, these peptides or epitopes are positioned within the N-terminal portion of CSP predominantly more N-terminal than the previously described hepatocyte receptor binding region (R1). Accordingly, PCMS based N-terminal CSP antigens are described and proposed for use in protocols to vaccinate against malaria or prevent or treat malaria.

In one embodiment, PCMS based N-terminal CSP antigens are described and proposed for use in protocols to induce antibodies against P. falciparum (Pf) sporozoites that effectively reduce the level of sporozoites. It is described herein that antibodies raised against NT CSP peptides induce or promote antibody dependent phagocytosis or complement mediated killing of sporozoites.

Further, in one embodiment that an enhanced PCMS response is induced with NT CSP peptides than with CT and/or NANP peptides. In one embodiment, a CSP NT peptide representing the CSP NT subregion of CSP and not (or substantially not) representing the CT or NANP central repeat region is co-administered with a second peptide representing C-terminal (CT) and/or central repeat peptides (NANP) subregions of CSP.

In one embodiment, a CSP NT peptide representing the CSP NT subregion of CSP and not representing the CT or NANP central repeat region is co-administered with a second or further peptide representing C-terminal (CT) and/or central repeat peptides (NANP) subregions of CSP wherein the peptide sequences are present together in the same antigen.

In one embodiment, co-administration of peptides may be simultaneously, such as where both peptides representing different sub-regions of PfCSP are present in the same administered composition, or where the peptides are separately administered either at the same time or sequentially over a period of time to induce an optimum immune response. The time period may be a day, week, fortnight, month or several months.

In one embodiment, a CSP NT peptide representing the CSP NT subregion of CSP comprises amino acids that present to the immune system of the subject an IgG antibody or PCMS inducing part of the NT subregion as described herein. In one embodiment, and for the avoidance of doubt, a CSP NT peptide does not comprise CT and/or NANP CSP subregions.

In one embodiment, the N-terminal PCMS antigen or peptide comprises DDGNNEDNEKLRKPKHKKLKQ (SEQ ID NO: 25) or ENWYSLKKNSRSLGENDDGNNEDNEKLRKPKHKKLKQPADG (SEQ ID NO: 57) or KQENWYSLKKNSRSLGENDDGNNEDNEKLRKPKHKKLKQPADGNPDP (SEQ ID NO: 1), or a peptide comprising SEQ ID NO:25 and N-terminal or C-terminal contiguous amino acids from the CSP NT sequences, SEQ ID NO: 57 or SEQ ID NO: 1, or a corresponding sequence from a different (non-3D7) strain or variant.

In one embodiment, antigens presenting the CT or NANP subregions of CSP such as those used in the RTS,S or R21 vaccines are employed in addition to NT CSP antigen/immunogen. While the present application describes PCMS antigens derived from Plasmodium falciparum, antigens comprising the same regions may be derived from other Plasmodium species, such as P. Vivax. Methods are also proposed for screening antigens for their capacity to stimulate PCMS. It is apparent to the skilled person how to put the present concept and invention into effect.

In one embodiment, the PCMS antigen comprises a PCMS peptide from the N-terminal amino acids 58 to 104 (SEQ ID NO: 1) of the CSP polypeptide. In one embodiment, the peptide comprises 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, or 47, 48 (or any derivable range therein) contiguous amino acids from the N-terminal amino acids 58 to 104 of the CSP polypeptide. In one embodiment, the peptide is set out in SEQ ID NO: 25 or 57.

In one embodiment, the PCMS antigen or NT CSP antigen further comprises a T-cell helper epitope.

In one embodiment, the PCMS peptide comprises 3, 4, 5, 6, 7, 8, 9, 10, 11, 12, 13, 14, 15, 16, 17, 18, 19, 20, 21, 22, 23, 24 contiguous amino acids from the N-terminal amino acids 58 to 81 (SEQ ID NO: 2) of the CSP polypeptide (amino acid numbering from illustrative P. falciparum strain 3D7).

In one embodiment, the PCMS peptide comprises 3, 4, 5, 6, 7, 8, 9, 10, 11, 12, 13, 14, 15, 16, 17, 18, 19, 20, 21, contiguous amino acids from the N-terminal amino acids 64 to 84 (SEQ ID NO: 3) of the CSP polypeptide

In one embodiment, the peptide comprises 3, 4, 5, 6, 7, 8, 9, 10, 11, 12, 13, 14, 15, 16, 17, 18, 19, 20, 21, 22, 23, 24 contiguous amino acids from the N-terminal amino acids 58 to 81 of the CSP polypeptide and additionally comprises 3, 4, 5, 6, 7, 8, 9, 10, 11, 12, 13, 14, 15, 16, 17, 18, 19, 20, 21, 22, 23, 24 contiguous amino acids from the N-terminal amino acids 82 to 104 (SEQ ID NO: 4) of the CSP polypeptide.

In one embodiment, the peptide comprises 3, 4, 5, 6, 7, 8, 9, 10, 11, 12, 13, 14, 15, 16, 17, 18, 19, 20, 21 contiguous amino acids from the N-terminal amino acids 64 to 84 of the CSP polypeptide wherein at least 3 contiguous amino acids include DDG or GNN, and wherein the peptide and additionally comprises 3, 4, 5, 6, 7, 8, 9, 10, 11, 12, 13, 14, 15, 16, 17, 18, 19, 20, 21, 22, 23, 24 contiguous amino acids from the N-terminal amino acids 76 to 100 (SEQ ID NO: 5) of the CSP polypeptide wherein at least 3 contiguous amino acids include KPK and not GNP. In one embodiment the PCMS peptide comprises 5 to 50 amino acids or 5 to 100 amino acids.

In one embodiment, the PCMS peptide sequence is at least 70, 72, 74, 76, 78, 80, 82, 84, 86, 88, 90, 92, 94, 96, 98, 99, or 100% identical with any one of SEQ ID NO: 1 to 5. In one embodiment the peptides sequence is at least 70, 72, 74, 76, 78, 80, 82, 84, 86, 88, 90, 92, 94, 96, 98, 99, or 100% identical with any one of SEQ ID NO: 1 to 5 and/or comprises at least one or more of the following contiguous amino acid sequences from 57 to 104: DDG (SEQ ID NO: 6), GNN (SEQ ID NO: 7), DDGN (SEQ ID NO: 8), DDGNN (SEQ ID NO: 9), DDGNNE (SEQ ID NO: 10), DDGNNED (SEQ ID NO: 11), DDGNNEDN (SEQ ID NO: 12), DDGNNEDNE (SEQ ID NO: 13), DDGNNEDNEK (SEQ ID NO: 14), DDGNNEDNEKL (SEQ ID NO: 15), DDGNNEDNEKLR (SEQ ID NO: 16), DDGNNEDNEKLRK (SEQ ID NO: 17), DDGNNEDNEKLRKP (SEQ ID NO: 18), DDGNNEDNEKLRKPK (SEQ ID NO: 19), DDGNNEDNEKLRRKPKH (SEQ ID NO: 20), DDGNNEDNEKLRKPKHK (SEQ ID NO:21), DDGNNEDNEKLRKPKHKK (SEQ ID NO: 22), DDGNNEDNEKLRKPKHKKL (SEQ ID NO: 23), DDGNNEDNEKLRKPKHKKLK (SEQ ID NO: 24), DDGNNEDNEKLRKPKHKKLKQ (SEQ ID NO: 25), DDGNNEDNEKLRKPKHKKLKQP (SEQ ID NO: 26), DDGNNEDNEKLRKPKHKKLKQPA (SEQ ID NO 27), and DDGNNEDNEKLRKPKHKKLKQPAD (SEQ ID NO 28).

In one embodiment the peptides sequence is at least 70, 72, 74, 76, 78, 80, 82, 84, 86, 88, 90, 92, 94, 96, 98, 99, or 100% identical with any one of SEQ ID NO: 1 to 5 and/or comprises at least one or more of the following contiguous amino acid sequences from 58 to 104: KQENWYSLKKNSRSLGENDDGNN (SEQ ID NO: 29), QENWYSLKKNSRSLGENDDGNN (SEQ ID NO: 30), KNSRSLGENDDGNN (SEQ ID NO: 31), NSRSLGENDDGNN (SEQ ID NO: 32), RSLGENDDGNN (SEQ ID NO: 33), SLGENDDGNN (SEQ ID NO: 34), LGENDDGNN (SEQ ID NO; 35), GENDDGNN (SEQ ID NO: 36), ENDDGNN (SEQ ID NO: 38), and NDDGNN SEQ ID NO: 39).

In one embodiment, C-terminal (CT) regions of CSP are absent from the subject PCMS antigen/peptide.

In another embodiment, C-terminal regions of CSP are present in the subject PCMS antigen.

In one aspect, the present application discloses that antibodies to the N-terminal region of CSP promoted a substantially higher relative phagocytic activity compared to antibodies directed to epitopes in the CT region. It is proposed in one embodiment that the CSP-based vaccine such as the RTS,S vaccine could be improved by the inclusion of PCMS peptides from the central region of the N-terminal domain. In another embodiment, PCMS antigen representing all or part of the N-terminal portion of CSP are presented to the immune system to stimulate an effective response in a subject. In one embodiment, the present application provides an immunogenic composition comprising a modified PfCSP or an N-terminal peptide fragment thereof comprising one or more N-terminal epitopes that stimulate an antibody response in a subject that stimulates sporozoite opsonisation and immune cell mediated clearance in a subject. Reference to a modified PfCSP means a non-full length CSP, and includes a truncated polypeptide compared to the naturally occurring polypeptide such as an N-terminally and/or C-terminally truncated polypeptide.

In one embodiment, the present application provides an immunogenic composition comprising a modified PfCSP, or an N-terminal peptide fragment thereof, each comprising one or more N-terminal epitopes that stimulate an antibody response in a subject that stimulates sporozoite opsonisation and immune cell mediated clearance in a subject. In one embodiment, the present application provides a polynucleotide construct encoding a modified PfCSP or an N-terminal peptide fragment thereof each comprising one or more N-terminal epitopes that stimulate an antibody response in a subject that stimulates sporozoite opsonisation and immune cell mediated clearance in a subject. Polynucleotides include, plasmids, vectors, expression vectors, RNA, DNA, hybrids and modified forms as described herein and known in the art. Polynucleotides may be used to express antigen in vitro or in vivo as known in the art.

In one embodiment, the present application provides a immunogenic composition comprising or encoding a modified PfCSP or an N-terminal peptide fragment thereof comprising one or more N-terminal epitopes that stimulate an antibody response in a subject that stimulates sporozoite opsonisation and immune cell mediated clearance in a subject and wherein the N-terminal fragment induces substantially N-terminal CSP specific antibodies that bind Fcγ-receptors on neutrophils and monocytes and induce antibody dependent opsonic phagocytosis of sporozoites or killing by natural killer cells.

In one embodiment, the modified PfCSP or PCMS antigen comprises amino acids 59 to 327 of CSP (SEQ ID NO:41) QENWYSLKKNSRSLGENDDGNNEDNEKLRKPKHKKLKQPADGNPDPNANPN VDPNANPNVDPNANPNVDPNANPNANPNANPNANPNANPNANPNANPNANP NANPNANPNANPNANPNANPNANPNANPNANPNANPNVDPNANPNANPNAN PNANPNANPNANPNANPNANPNANPNANPNANPNANPNANPNANPNANPNA NPNANPNANPNKNNQGNGQGHNMPNDPNRNVDENANANSAVKNNNNEEPS DKHIKEYLNKIQNSLSTEWSPCSVTCGNGIQVRIKPGSANKPKDELDYANDIEK KICKMEKCSSVFNVVNSGS.

In one embodiment the PCMS antigen comprises one or more of peptides 11 to 23 in FIG. 14 (SEQ ID NO: 9 to 21). In particular, one or more of peptides 13 to 15 or a peptide that shares contiguous sequences/epitopes within peptides 13 to 15. In particular peptides 17 to 20 or shared contiguous sequences/epitopes within peptides 13 to 15. Epitope mapping is known in the art. The specification describes methods to determine the ability of an antigen to promote PCMS. In one embodiment of the present application such N-terminal portions or PCMS epitopes are unexpectedly recognised by antibodies to the N-terminal amino acids 58 to 104, and at least a proportion of which stimulate sporozoite killing through phagocytosis or complement mediated mechanisms.

Accordingly, one embodiment the present application provides a PCMS antigen as described above that elicits antibodies in a mammalian subject to the N-terminal portion of CSP (comprising amino acids 19 to 104 (3D7. In one embodiment, antibodies generated include those that stimulate Plasmodium sporozoite killing through phagocytosis or complement mediated mechanisms.

In one embodiment is provide compositions comprising a nucleic acid molecule encoding a PCMS antigen as described herein. The gene ID for P. falciparum CSP (3D7/NF54 strain) is PF3D7_0304600. Details on the sequences of P. falciparum CSP is available at PlasmoDB (plasmodium genomics resource) (plasmodb.org/plasmo/app/record/gene/PF3D7_0304600).

In another embodiment, a viral or non-viral vector, or viral like particle comprising the PCMS antigen encoding sequences is provided.

In one embodiment, there is provided a composition comprising or encoding a PCMS antigen as described herein.

In one embodiment the present application enables a pharmaceutical composition comprising or encoding a PCMS antigen as described herein. In one embodiment the composition comprises a pharmaceutically acceptable diluent and/or carrier. In one embodiment the carrier is a nanocarrier. Suitable nanocarriers are described in Kurtovic et al Frontiers in Immunology Vol. 12, Article 641421 March 2021, incorporated herein. In one embodiment, the composition may comprise an adjuvant.

In one embodiment, combinations of CSP vaccine antigens may be administered in combination, at the same time or sequentially. For example, C-terminal CSP-based antigens and the present N-terminal PCMS based antigens may be administered together at the same time or spaced apart, in the same or different compositions or vectors.

In one embodiment, CSP-based antigens representing CT and NANP subregions of CSP include the RTS,S and/or R21 vaccine candidates.

In another aspect the present application provides a method of treating or preventing malaria or inducing a functional (eg, PCMS) immune response, comprising administering to a subject an effective amount of a composition comprising the peptide antigens or their encoding sequence as herein described.

In one embodiment, the composition comprising or encoding all or part of the NT region, (such as the N-terminal only PCMS based antigen) also comprises or encodes antigen from the central or CT region of CSP. In one embodiment, the different antigens are administered at the same time or spaced apart in the same or different compositions. As illustrated in FIG. 15, vaccination studies demonstrate that a mixture of the RTS,S immunogen (comprising CT and NANP subregions) and the NT CSP peptide immunogen effectively generates IgG to all 3 regions of CSP.

In another embodiment, the present application provides a composition as described herein for use or when used in treating or preventing a Plasmodium infection.

In another embodiment, the present invention provides for the use of a PCMS antigen or peptide or composition as described herein in the manufacture of a medicament for treating or preventing a Plasmodium infection or malaria in a subject.

Any method or composition of the present disclosure can consist of or consist essentially of—rather than comprise/include/contain/have—any of the described elements and/or features and/or steps. Thus, in any of the claims, the term “consisting of” or “consisting essentially of” can be substituted for any of the open-ended linking verbs recited above, in order to change the scope of a given claim from what it would otherwise be using the open-ended linking verb. A composition “consisting essentially of” the recited elements excludes any further active ingredients but does not exclude pharmaceutical excipients, buffers, structural components, etc.

This application claims the benefit of priority from Australian Provisional Patent Application 2020902779 filed 6 Aug. 2020, the entire contents of which are incorporated herein by reference.

The following numbered clauses are also encompassed.
1. An immunogenic or vaccine composition comprising a PCMS antigen that promotes antibody dependent Phagocytosis or Complement Mediated killing of Sporozoites (PCMS), the PCMS antigen comprising a PCMS peptide comprising the N-terminal amino acids 58 to 104 (SEQ ID NO: 1) of the P. falciparum CSP polypeptide or a PCMS peptide of 3 to 48 contiguous amino acids from the N-terminal amino acids 58 to 104 (SEQ ID NO: 1) of the P. falciparum Circumsporozoite polypeptide (CSP) polypeptide of strain 3D7 or a corresponding peptide from a different P.falciparum strain/isolate.
2. The composition of claim 1, wherein the PCMS peptide comprises N-terminal amino acids 58 to 81 (SEQ ID NO: 2) of the CSP polypeptide or 3 to 24 contiguous amino acids from the N-terminal amino acids 58 to 81 (SEQ ID NO: 2) of the CSP polypeptide.
3. The composition of claim 2, wherein the PCMS peptide comprises the N-terminal amino acids 64 to 84 (SEQ ID NO: 3) of the CSP polypeptide or 3 to 21 contiguous amino acids from the N-terminal amino acids 64 to 84 (SEQ ID NO: 3) of the CSP polypeptide.
4. The composition of claim 1 or 2, wherein PCMS peptide comprises 3 to 24 contiguous amino acids from the N-terminal amino acids 58 to 81 of the CSP polypeptide and additionally comprises 3 to 24 contiguous amino acids from the N-terminal amino acids 82 to 104 (SEQ ID NO: 4) of the CSP polypeptide.
5. The composition of claim 1 or 3, wherein the PCMS peptide comprises 3 to 21 contiguous amino acids from the N-terminal amino acids 64 to 84 of the CSP polypeptide wherein at least 3 contiguous amino acids include DDG or GNN, and wherein the peptide and additionally comprises 3 to 24 contiguous amino acids from the N-terminal amino acids 76 to 100 (SEQ ID NO: 5) of the CSP polypeptide wherein at least 3 contiguous amino acids include KPK and not GNP.
6. The composition of any one of claims 1 to 5, wherein the PCMS peptide comprises 5 to 50 amino acids or 10 to 50 or 20 to 50, or 5 to 100 amino acids.
7. An immunogenic or vaccine composition comprising a PCMS antigen that promotes antibody dependent Phagocytosis or Complement Mediated killing of Sporozoites (PCMS), the PCMS antigen comprising a PCMS peptide sequence having at least 70 to 100% identity with any one of SEQ ID NO: 1 to 5, and/or comprises at least one or more of the following contiguous amino acid sequences from amino acids 57 to 104 (SEQ ID NO: 1): DDG, GNN, DDGN, DDGNN, DDGNNE, DDGNNED, DDGNNEDN, DDGNNEDNE, DDGNNEDNEK, DDGNNEDNEKL, DDGNNEDNEKLR, DDGNNEDNEKLRK, DDGNNEDNEKLRKP, DDGNNEDNEKLRKPK, DDGNNEDNEKLRRKPKH, DDGNNEDNEKLRKPKHK, DDGNNEDNEKLRKPKHKLK, DDGNNEDNEKLRKPKHKL, DDGNNEDNEKLRKPKHKLKQ, DDGNNEDNEKLRKPKHKLKQP, DDGNNEDNEKLRKPKHKLKQPA, and DDGNNEDNEKLRKPKHKLKQPAD.
8. The composition of claim 7, wherein the PCMS peptide sequence is at least 70 to 100% identical with any one of SEQ ID NO: 1 to 5 and/or comprises at least one or more of the following contiguous amino acid sequences from 58 to 104 (SEQ ID NO: 1): KQENWYSLKKNSRSLGENDDGNN, QENWYSLKKNSRSLGENDDGNN, KNSRSLGENDDGNN, NSRSLGENDDGNN, RSLGENDDGNN, SLGENDDGNN, SLGENDDGNN, LGENDDGNN, GENDDGNN, ENDDGNN, and NDDGNN.
9. The composition of any one of claims 1 to 8, wherein C-terminal (CT) regions of CSP are absent from the subject PCMS antigen.
10. The composition of any one of claims 1 to 8, wherein the PCMS antigen or the composition further comprises C-terminal (CT) regions of CSP.
11. An immunogenic composition comprising or encoding a modified PfCSP or an N-terminal peptide fragment thereof comprising one or more N-terminal epitopes that stimulate an antibody response in a subject that stimulates sporozoite opsonisation and immune cell mediated clearance in a subject, wherein the N-terminal fragment induces substantially N-terminal CSP specific antibodies that bind Fcγ-receptors on neutrophils and monocytes and induce antibody dependent opsonic phagocytosis of sporozoites or killing by natural killer cells.
12. The composition of claim 11, wherein the modified PfCSP or PCMS antigen comprises amino acids 59 to 327 of CSP (SEQ ID NO:6) QENWYSLKKNSRSLGENDDGNNEDNEKLRKPKHKKLKQPADGNPDPNANPN VDPNANPNVDPNANPNVDPNANPNANPNANPNANPNANPNANPNANPNANP NANPNANPNANPNANPNANPNANPNANPNANPNANPNVDPNANPNANPNAN PNANPNANPNANPNANPNANPNANPNANPNANPNANPNANPNANPNANPNA NPNANPNANPNKNNQGNGQGHNMPNDPNRNVDENANANSAVKNNNNEEPS DKHIKEYLNKIQNSLSTEWSPCSVTCGNGIQVRIKPGSANKPKDELDYANDIEK KICKMEKCSSVFNVVNSGS.
13. The composition of claim 12, wherein the PCMS antigen comprises one or more of peptides 11 to 23 in FIG. 14 (SEQ ID NO: 7 to 19).

Methods used in the present application are described herein below without limitation.

### Recombinant Proteins and Synthetic Peptide Antigens

Sequence selection and modification: Recombinant proteins were generated based on the 3D7 allele P. falciparum CSP (XP_001351122), expressed in the mammalian HEK293F cell line. CSP contains 397 amino acids and can be divided into several domains: The N-terminal signal peptide (amino acids 1-18), the N-terminal (NT) domain (amino acids 19-104) that contains a free cysteine residue at position 25, the central repeat region consisting of 4 NVDP and 38 NANP repeats (amino acids 105-272), and the C-terminal domain (amino acids 273-398) that contains a predicted GPI anchor Omega site at Cys-374.

In one aspect, different subunits of CSP representing the C-terminal region (CT) and a fusion between the N-terminal and C-terminal regions (N+C; NANP and NVNP repeats absent). The CT construct contains amino acids 273-383 and represents a slightly truncated form of the CT with a disrupted GPI anchor motif. The N+C construct contains a slightly truncated form of the N-terminus (amino acids 26-104; removing the endogenous signal sequence), which is fused to the C-terminal domain (amino acids 273-383) via a short GGGS linker. To mediate secretion of these recombinant proteins into media and purification via nickel resin chromatography, a signal peptide for Tissue Plasminogen Activator (TPA), followed by a 6-histidine tag was fused to the N-terminus of both proteins. Both protein sequences were then assessed for potential glycosylation sites but none were identified and so no further modifications were made to the protein sequences.

Synthetic peptides were generated to represent the N-terminal region (amino acid 19-104, NT) and the central repeat region (NANP×15, NANP) of CSP (LifeTein, NJ, USA).

Generation of protein expression vectors: The CT and N+C protein sequences were used to generate DNA sequences that were codon optimized for mammalian expression and then synthesized (GeneArt, Thermo Fisher Scientific). The synthetic genes were supplied in a Puc vector, and then cloned into pcDNA 3.1+ using Xho1 and BamH1 restriction sites. The final plasmids were quantified and used to transfect HEK293F cells.

HEK293F cell culture and transfection: HEK293 Freestyle™ cells (Thermo Fisher Scientific) were cultured following the manufacturer's protocols. In brief, cells were cultured in Erlenmeyer shaker flasks (125 ml, Corning) with FreeStyle™ 293 Expression Medium (Thermo Fisher Scientific) at 37° ° C., 8% CO₂ at 135×rpm on an orbital shaker. Cells were counted using the trypan blue (0.4%; Thermo Fisher Scientific) cell exclusion method using Countess™ Cell Counting Chamber Slides (Thermo Fisher Scientific) and the Countess™ automated cell counter (Thermo Fisher Scientific). HEK293F cells were transfected for protein expression following the manufacturer's protocol (Thermo Fisher Scientific) with minor alterations. On the day of transfection, cells were centrifuged (700 g at 4° C., 10 min) and resuspended in HEK293F expression media with 1:100 antibiotic/anti-mycotic solution (Thermo Fisher Scientific) at a final density of 1×10⁶ cells/ml. For a 30 ml transfection, 90 μl of Polyethylenimine (PEI) transfection reagent (25 kDA linear; Polysciences; stock 1 mg/ml) was added to 0.6 ml of OptiPro™ Serum Free Medium (Thermo Fisher Scientific) and incubated for 5 minutes. This solution was then added to the DNA solution (30 μg of purified plasmids and 0.6 ml of OptiPro™ Serum Free Medium (Thermo Fisher Scientific). After incubating for 10 min at room temperature, this final solution was added to the cells, and then returned to the orbital shaker and incubated. The next day, Lupin (1:40 of 20% w/v, Biotech Solabia) and Pluronic acid F-68 (1:100 of 10% w/v) (Thermo Fisher Scientific) were added. The expressed proteins were harvested 6 days post-transfection by centrifuging cells (700×g, minutes) to collect supernatant that was then filtered (0.2 μm membrane) and stored at 4° C. until purification. Protein expression was tested for using SDS-PAGE gels and western blot.

Protein purification and dialysis: Harvested media containing the expressed proteins was passed over nickel resin columns (Life Technologies, Thermo Fisher Scientific), washed with 20 mM imidazole/PBS (Sigma-Aldrich), and bound proteins were eluted in several 1 ml fractions in 500 mM imidazole/PBS. Eluted fractions were tested for the presence of protein by spectroscopy and SDS-PAGE. Fractions containing protein were pooled, filter sterilized, dialyzed into sterile PBS, and adjusted to a concentration of 1 mg/ml via centrifugation using 10,000MW cut-off filters. Protein size and purity was confirmed by SDS-PAGE.

### Generation of Antibodies to Different CSP Proteins

Briefly, rabbits (Oryctolagus cuniculus) (n=2) were immunised with one of the following antigens: full-length CSP, N+C recombinant protein, and NT peptide. Each rabbit received three doses of antigen (200 μg/dose) co-administered with Freud's adjuvant on day 0, day 28 and day 56. Rabbit sera were collected after the third immunization on day 68, and rabbit IgG were purified by protein A positive selection. The rabbit anti-N+C IgG were initially tested, and shown to predominantly recognise the C-terminal region of CSP, and not the N-terminal region (FIG. 12). The rabbit anti-NT IgG was confirmed to recognise the N-terminal region of CSP (FIG. 11). Both NT and CT antibodies reacted with full length CSP.

Isolation of PBMC, Neutrophils and Whole Leukocytes from Peripheral Blood

Peripheral blood mononuclear cell (PBMC) and neutrophils were isolated from peripheral blood using previously established methods (Quinn et al, 2007). Briefly, peripheral blood from healthy donors were separated using Ficoll gradient centrifugation. The Buffy layers which contain PBMCs and the RBC pellet which contains neutrophils were separately collected. For PBMC isolation, cells collected from the Buffy layer were washed 4 times with PBS-1% NCS at 4° C. PBMCs were subsequently resuspended in RPMI1640-10% fetal bovine serum (FCS) and kept on ice. Neutrophils were enriched by dextran segmentation from the RBC pellet post Ficoll gradient centrifugation, followed by hypotonic lysis. Whole leukocytes were isolated from whole blood by initial enrichment with dextran segmentation followed by hypotonic lysis. Isolated neutrophils and whole leukocytes were adjusted to 5×10⁵ neutrophil/ml or 5×10⁶ phagocytes (monocytes & neutrophils)/ml and subsequently resuspended in RPMI1640 supplemented with 10% FCS and 2.5% heat inactivated human serum and kept on ice. Cells were phenotyped for FcγR expression using antibodies to FcγRI (Clone 10.1 BD Bioscience), FcγRIIa (Clone 8.26, BD Bioscience), and FcγRIII (Clone 3G8 BD Bioscience). Antibodies to CD14 (Clone M5E2, BD Bioscience) was also use to determine monocyte subsets.

### Maintenance of THP-1 Cells

Cells of the THP-1 promonocytic cell line were maintained in RPMI-1640 with 0.002 mol/L L-glutamine, 0.01 mol/L HEPES, and 10% fetal calf serum (FCS). Cell density was monitored closely and maintained between 1×10⁵ and 1×10⁶ cell/ml. Cells were passaged every 6 days or when cell density approached 1×10⁶ cells/ml.

Culture and Isolation of P. berghei Sporozoites

Anopheles stephensi mosquitoes were fed on Swiss Webster mice infected with a chimeric CSP expressing P. berghei ANKA parasite line (Triller et al, 2017). Salivary glands were dissected 18-22 days post the infectious blood meal and homogenized in PBS. The sporozoites were passed through a 70 μm tissue strainer (Falcon) and counted using a haemocytometer.

Culture and Isolation of P. falciparum Merozoites

The D10 line of P. falciparum were maintained in RPMI-HEPES supplemented with 0.5% Albumax (Life Technologies) and 0.18% NaHCO₃. Cultures were maintained below 10% parasitemia and synchronized by sorbitol treatment. The merozoites were isolated using previously published methods (Osier, 2014). Briefly, schizoint stage of the infected erythrocytes were enriched by magnetic purification and continued cultured in medium supplemented with transepoxysuccinyl-L-leucylamido(4-guanidino) butane (E64, Sigma-Aldrich) for 8 hours before passing the mature schizoints through a 1.2 um filter to release the merozoites. The merozoites were adjusted to 5×10⁷/ml for opsonic phagocytosis assay or 1×10⁷/ml for coating to an ELISA plate for FcγR binding assay.

### Covalent Coupling of Antigens to Fluorescent Latex Beads

Amine-modified fluorescent latex beads of 2.0 μm size (Sigma-Aldrich) were washed twice with 400 μl of PBS and centrifuged at 2000×g for 3 minutes. Glutaraldehyde of 8% in PBS was added to the beads and incubated on a roller overnight at 4° C. After washing with PBS, 1 mg/ml of recombinant CSP was added to the beads and incubated on a vortex for 6 hours. Subsequently, the beads were centrifuged and resuspended in 200 μl of ethanolamine and incubated for 30 minutes on a vortex to quench all the remaining amine groups. The beads were subsequently washed in PBS and blocked with 1% BSA overnight at 4° ° C. The antigen-coated beads were kept in a sonicating water bath for 20 minutes at 4° C. to reduce aggregation and subsequently adjusted to 5×10⁷ beads/ml. The beads were stored at 4° C. in the presence of 0.1% SDS and 0.02% NaN3.

### Opsonic Phagocytosis Assays

Opsonic phagocytosis assays were performed in variable settings for different purposes as described below and in Table 1.

Opsonic phagocytosis of P. falciparum and P. berghei sporozoites using isolated cells: Cryopreserved P. falciparum sporozoites were provided by PATH-MVI (Sanaria, Rockville, USA) and stored in liquid nitrogen, and were thawed at 37° ° C. for 40 seconds as described in the manufacture's user manual. Freshly dissected P. berghei sporozoites that were a chimera expressing the PfCSP (see for example Triller, 2017). The sporozoites were stained with 10 μg/ml ethidium bromide for 1 hour on ice followed by 3 washes with RPMI-HEPES. For opsonisation, 50,000 sporozoites were incubated with 1 ul of test serum for 1 hour on ice, followed by co-incubation with 5,000 neutrophils at concentration of 5×10⁵ cells/ml in RPMI-1640 supplemented with 10% FCS and 2.5% heat-inactivated human serum from malaria-naïve Melbourne donors. An additional 1 ul of test serum were also added to account for the volume change from adding neutrophils and maintain the same test antibody concentration during phagocytosis. The co-incubation was allowed for 15 minutes at 37° ° C. in a 5% CO₂ incubator for phagocytosis to occur. Cells were centrifuged at 4° C. and washed with cold PBS-1% NCS-0.04% NaN₃ (FACS buffer) to stop phagocytosis activity. The level of phagocytosis was quantified by flow cytometry (FACS CantoII, BD Biosciences) and analysed using FlowJo software. The data were presented as phagocytosis index (PI), which was defined as the proportion of neutrophils or THP-1 cells that had phagocytosed sporozoites. For experiments on the Kanyawegi and Chulaimbo cohort, PI was further standardized as relative phagocytosis index (RPI), which was defined as the percentage of PI for each sample in relative to the positive control. Unopsonised sporozoites were included as negative controls in all assays. Standard neutrophil phagocytosis assays included 2.5% human serum from malaria non-exposed donors. Standard THP-1 phagocytosis assays were performed using established methods that include 10% FCS.

Phagocytosis of antigen-coated beads or merozoites using isolated cells: The phagocytosis assay was adapted from previously published methods with modifications²⁸. Briefly, 1×10⁶ antigen coated fluorescent latex beads or merozoites were opsonised with serum samples for 1 hour. The beads or merozoites were washed thrice with RPMI-1640 before co-incubation with 1×10⁵ neutrophils for phagocytosis. Phagocytosis was allowed to occur for 20 minutes for beads or 10 minutes for merozoites at 37° C. and the cells were subsequently washed with FACS buffer at 300 g and 4° C. for 4 minutes. The proportion of neutrophils containing fluorescent beads or merozoites was evaluated by flow cytometry (FACS CantoII, BD Biosciences) and analysed using FlowJo software. In some assays, the THP-1 cell line was used with standard conditions that include 10% FCS; in other assays additional 2.5% human serum from malaria naïve Melbourne donors was used for comparison.

Phagocytosis of antigen-coated beads or P. berghei sporozoites using whole blood leukocyte preparations: The CSP-coated beads were opsonised with a rabbit polyclonal antibody raised against CSP. Phagocytosis was performed by co-incubating the opsonised beads with the total leukocyte fraction from whole blood containing 1×10⁶ phagocytes (monocytes and neutrophils) for 20 minutes at 37° C. in a 5% CO₂ incubator. These experiments were performed using variable antibody concentrations for opsonisation or variable bead to phagocyte ratios. For testing opsonic phagocytosis of P. berghei sporozoites by whole leukocyte preparations, 5×10⁵ sporozoites were opsonised with a pool of malaria exposed immune adult sera at dilution of 1:100, followed by co-incubation with whole leukocytes containing 5×10⁴ phagocytes. The cells were centrifuged at 4° C. for 4 minutes to stop phagocytic activity and different phagocytes (monocytes and neutrophils) were labelled with fluoresce conjugated antibodies for further analysis. Monocytes were labelled with anti-CD14-Alexa657 (MφP9 BD Bioscience) and anti-CD16-BV421 (3G8 BD Bioscience) and neutrophils were labelled with anti-CD66b-APC-H7 (G10F5 BD Bioscience). For FACS analysis, phagocytosed beads were selected as PE^{high} and FSC^{high} population. Beads that were phagocytosed by monocytes and neutrophils were further distinguished using CD14 and CD66b staining.

The number of beads phagocytosed by monocytes and neutrophils were calculated based on the fluorescence intensity. This number was further standardized using the total number of phagocytes (monocytes and neutrophils) and expressed as beads per 100 phagocytes, representing the phagocytosis rate of different cell types.

Inhibiting opsonic phagocytosis by blocking Fcγ-receptors: Different FcγRs were blocked using specific blocking antibodies. FcγRI was blocked with 50 μg/ml blocking antibody (mAb 10.1 Merck), FcγRIIa was blocked with 50 μg/ml blocking antibody (mAb IV.3), FcγRIII was blocked with 50 μg/ml blocking antibody (mAb 3G8). THP-1 cells and neutrophils were treated with each FcγR blocker respectively at 4° C. for 30 minutes before co-incubation with CSP-coated latex beads, which have been opsonised with serum from immune adults. Phagocytosis assay was performed as mentioned above.

### Measuring Total IgG and IgG Subclasses by ELISA

Total IgG and IgG subclasses to recombinant proteins were measured by ELISA as previously described (Kurtovic, L., et al. Human antibodies activate complement against Plasmodium falciparum sporozoites, and are associated with protection against malaria in children. BMC Med 16, 61 (2018). Briefly, CSP was coated at 1 μg/ml on 96-well Maxisorp microtiter plates (NUNC, Roskilde, Denmark,) and incubated overnight at 4° C. Plates were washed with PBS containing 0.05% Tween20 (PBS-Tween) and blocked with PBS-1% BSA at 37° C. for 2 hours. Human serum or rabbit IgG were diluted to the desired concentration and added to the plates in duplicates. For total IgG detection, HRP-conjugated goat-anti-human IgG was used at 1:2500 dilution followed by ABTS substrate for colour development. The reaction was stopped with 1% SDS and absorbance was measured at 405 nm. For IgG subclass detection, monoclonal antibodies to human IgG1, IgG2, IgG3 and IgG4 were used at 1:1000 dilution, followed by HRP conjugated goat anti-mouse IgG used at 1:2500 dilution. Finally, 3,3′,5,5′-Tetramethylbenzidine (TMB, Life Technologies) substrate was used for colour development, and the reaction was stopped with of 1M H₂SO₄. The absorbance was measured at 450 nm. A pool of immune adult serum with high IgG reactivity to CSP was used as positive control.

### Fcγ Receptor-Binding Assay

The Fcγ receptor binding assay was adopted from previously published methods with minor modifications (Wines, B. D., et al. Dimeric FcgammaR Ectodomains as Probes of the Fc Receptor Function of Anti-Influenza Virus IgG. J Immunol 197, 1507-1516 (2016)) See Lichtfuss, G. F. et al. HIV inhibits early signal transduction events triggered by CD16 cross-linking on NK cells, which are important for antibody-dependent cellular cytotoxicity. J. Leukoc. Biol. 89, 149-158 (2011). Briefly, 50 μl of recombinant CSP at concentration of 1 μg/ml was coated on Maxisorp™ plates and incubated overnight at 4° C., followed by 3 washes with PBS-Tween. The plates were then blocked with 200 μl of 1% BSA-PBS at 37° C. for 2 hours followed by 3 washes with PBS-Tween. Serum samples were diluted at 1:50 in PBS-BSA and 50 μl of each sample was added to the plates in duplicate and incubated at room temperature for 2 hours, followed by 3 washes in PBS-Tween. Fifty microliter of biotin-conjugated recombinant soluble FcγRIIa H131 ectodomain dimers (0.2 μg/ml) or FcγRIII V158 ectodomain dimers (0.1 μg/ml) was added to each well and incubated at 37° C. for 1 hour followed by 3 washes with PBS-Tween. This was followed by a secondary HRP conjugated streptavidin (Thermo Fisher Scientific, 1:10,000) in PBS-BSA at 37° C. for 1 hour followed by 3 washes with PBS-Tween. Finally, 50 μl of TMB liquid substrate was added for 20 minutes to measure enzymatic reactivity. The reaction was stopped with 50 μl of 1M H₂SO₄ solution. The level of binding was measured as optical density at 450 nm. Pooled human IgG from malaria-exposed immune adults (1/100) and rabbit polyclonal IgG to CSP (1:500) was used as positive controls. Individual sera from naive Melbourne adults (1/50) were used as negative controls. For some assays, the serum samples were pre-treated with NT peptide at concentration of 20 μg/ml for 2 hours prior incubation in the antigen coated plate to deplete NT region specific antibodies.

For FcγR binding to merozoites, 50 μl of merozoites at a concentration of 1×10⁷/ml were coated to Maxisorp™ plates. The FcγR binding assay were performed as described above, except that PBS without 0.05% Tween were used for washing and purified human IgG from malaria exposed immune adults²⁸ at concentration of 1/250 were used for positive controls.

### Antibody-Dependent Cell-Mediated Cytotoxicity Assay

ADCC-inducing activity of a selection of serum samples was assessed using the ADCC Reporter Bioassay kit (Promega, Cat. No. G7010) as per the manufacturer's instructions, with the following modifications. CSP coated beads were opsonised with human serum at 1:50 dilution. Reporter cells were plated at 72,000 cells per well together with CSP coated fluorescent beads at a 10:1 ratio. After 6 hours of incubation, chemiluminescence substrate was added and luminescence detected and quantified approximately every 5 minutes for 40 minutes (CLARIOstar, BMG LabTech). Data presented were recorded about 30 minutes after the addition of the chemiluminescence substrate.

The level of ADCC was also confirmed in primary NK cells using a previous published method (Lichtfuss, G. F., et al. HIV inhibits early signal transduction events triggered by CD16 cross-linking on NK cells, which are important for antibody-dependent cellular cytotoxicity. J Leukoc Biol 89, 149-158 (2011)). Briefly, PBMCs containing NK cells were cultured in RPMI1640 supplemented with 10% FCS and 100 IU/ml of interleukin-2 (IL-2) overnight. CSP coated beads were opsonised with human serum at 1:50 dilution, then co-cultured with the primed PBMCs in the present of anti-CD107a-AF647 (H4A3, BD Bioscience) for 1 hour. Subsequently, brefeldin A (Sigma-Aldrich) and protein transport inhibitor (BD Bioscience) were added to the co-culture at concentration of 5 μg/ml and 1/1500 respectively and continued to incubate for 3 hours. After incubation, the cells were centrifuged at 300×g for 4 minutes at 4° C. and stained with anti-CD3-APC-H7 (SK7, BD Bioscience), anti-CD56-PE-Cy7 (B159, BD Bioscience) and anti-CD16-BV450 (3G8, BD Bioscience). NK cells were defined as CD3, CD56+ lymphocytes and the level of ADCC were quantified as percentage of NK cells with CD107a staining by flow cytometry (LSR Fortessa X-20, BD Bioscience).

### Data Analysis

Statistical analyses were performed using Prism version 7 (GraphPad Software Inc) and STATA version 13.1 (STATACorp). Statistics test and p values are indicated in figure legends. Correlations between phagocytosis, FcγR binding and IgG levels were evaluated using Spearman's correlation coefficients and multiple linear regression models. The differences in levels of phagocytosis, FcγR binding and IgG levels across age groups and different opsonization conditions were assessed using the one-way ANOVA with multiple comparisons. To evaluate the antibody functional activities irrespective of total IgG titer to CSP, FcγR binding efficiencies and phagocytosis efficiencies were calculated as the level of FcγR binding or the level of opsonic phagocytosis in relative to total IgG titer to CSP. Spearman's correlation and Mann-Whitney test were used to analyse the antibody functional efficiencies among children and adults. Two-way ANOVA were used to compare the opsonic phagocytosis by neutrophils and monocytes in the whole leukocyte assays. Kruskal-Wallis test and Mann-Whitney test were performed for data that were not normally distributed. Samples were classified as positive for antibody if reactivity was greater than the mean+3 standard deviations of the values of the malaria non-exposed controls.

### Mapping Antibody Epitopes of the NT Region

An array containing 70 biotinylated 15-mer peptides representing the linear sequence of CSP (3D7/NF54 strain) was synthesized (Mimotopes, Australia). Each peptide had a short SGSG spacer between the CSP peptide sequence and the biotin tag (which was added to aid immobilisation), and a 12aa overlap between peptides. Antibody assays were conducted using previously established approaches (Feng et al. 2018; Boyle et al. 2015); 50 μl per well of peptides were added to ELISA plates that were pre-coated with 1 ug/ml streptavidin (Sigma-Aldrich). Saturation of peptide binding was achieved and confirmed by quantification of residual biotin binding sites on streptavidin using biotin-HRP. followed by blocking with 10% milk, and washing with PBS with Tween 0.05%. Rabbit polyclonal IgG raised against the NT region of CSP was added to each well at concentration of 1 in 500. The level of rabbit IgG binding was detected by HRP conjugated goat anti-rabbit IgG (Millipore) and quantified using the TMB substrate.

The present description is further illustrated by the following examples, which should not be construed as limiting in any way.

### EXAMPLES

### Example 1—Opsonic Phagocytosis of Sporozoites is Predominantly Mediated by Neutrophils in Peripheral Blood

To investigate the relevance of FcγR-mediated mechanisms against sporozoites, opsonic phagocytosis assays were established using the undifferentiated THP-1 cell line (pro-monocytic cells), under standard conditions (Osier, F. H., et al. Opsonic phagocytosis of Plasmodium falciparum merozoites: mechanism in human immunity and a correlate of protection against malaria. BMC Med 12, 108 (2014); Steel, R. W., et al. An Opsonic Phagocytosis Assay for Plasmodium falciparum Sporozoites. Clin Vaccine Immunol 24(2017). Serum antibodies from immune adults were used who were resident in a region with high malaria endemicity and have significant clinical immunity to malaria and antibodies to CSP (Kurtovic, L., et al. Human antibodies activate complement against Plasmodium falciparum sporozoites, and are associated with protection against malaria in children. BMC Med 16, 61 (2018).

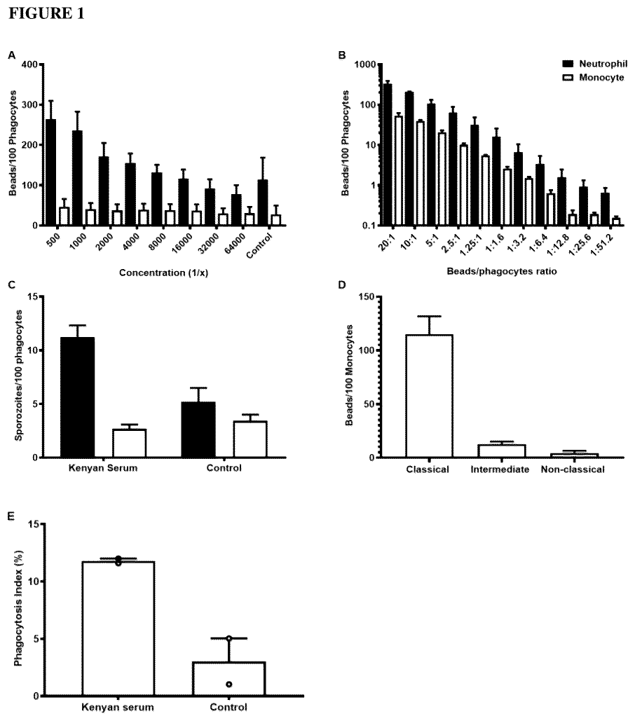

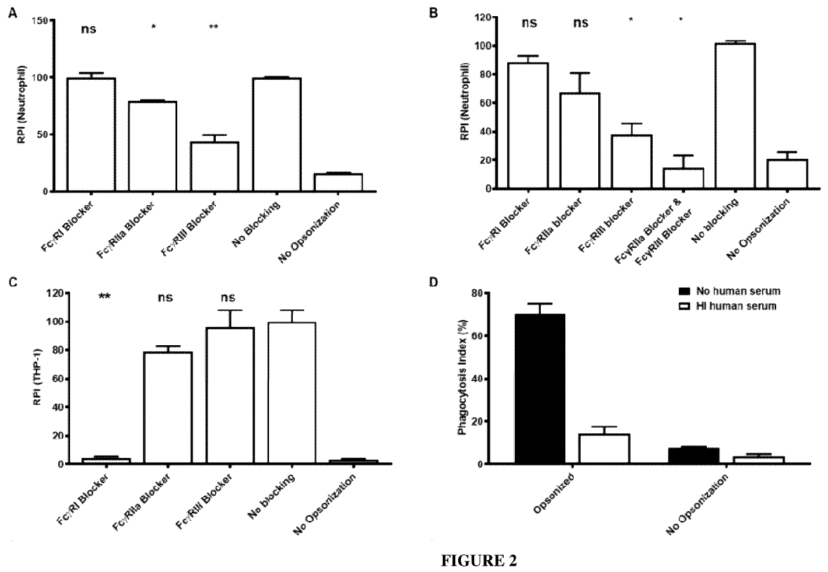

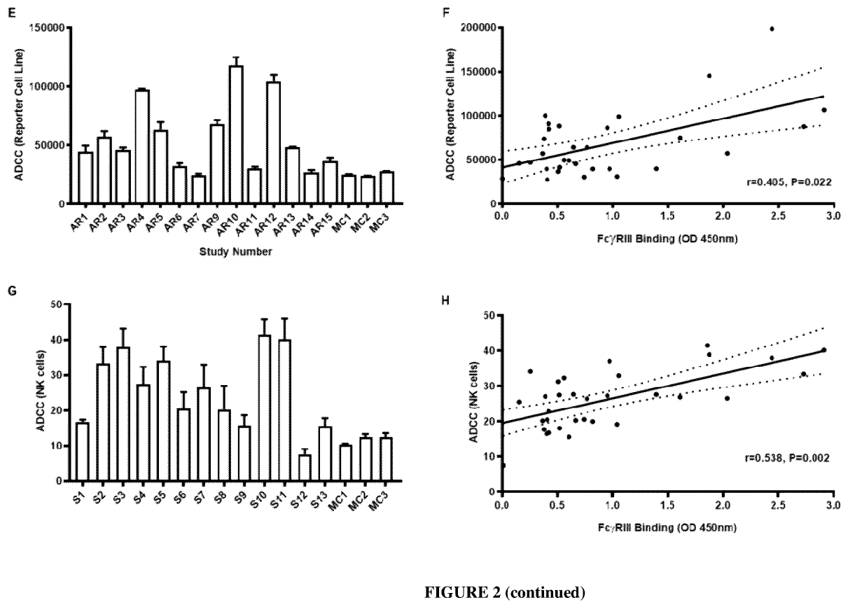

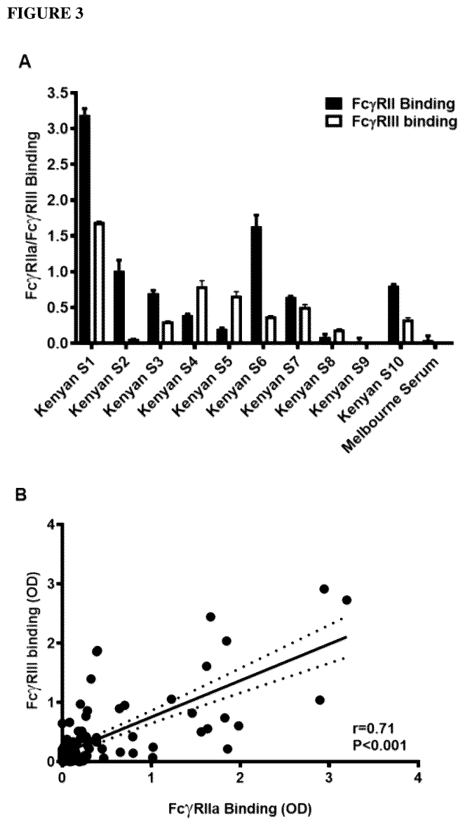

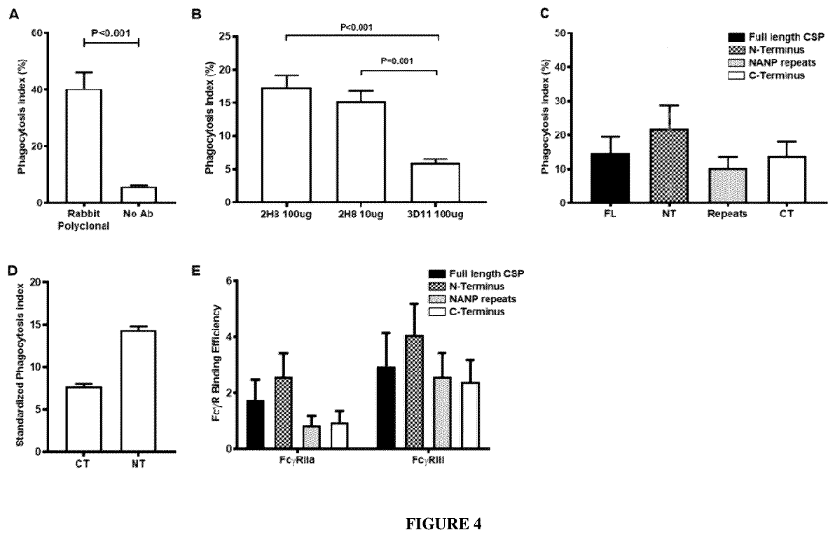

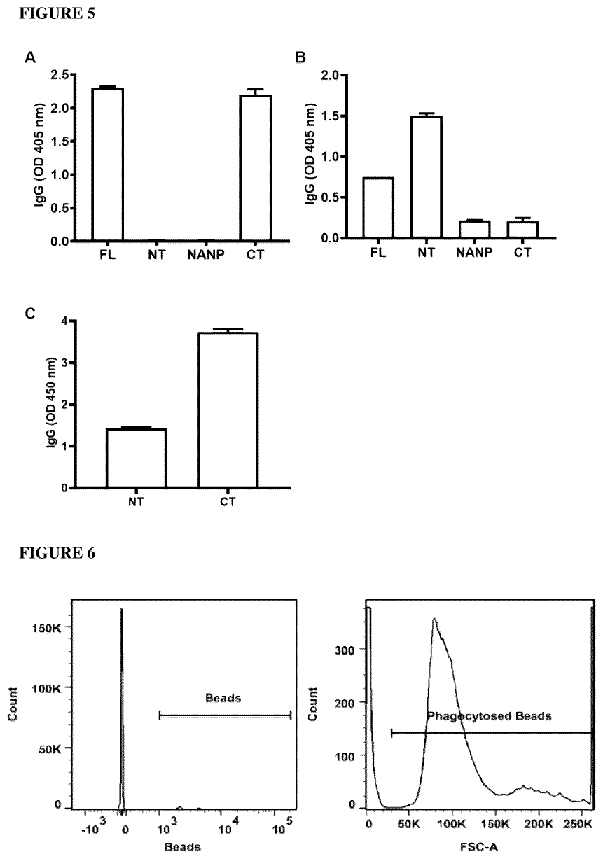

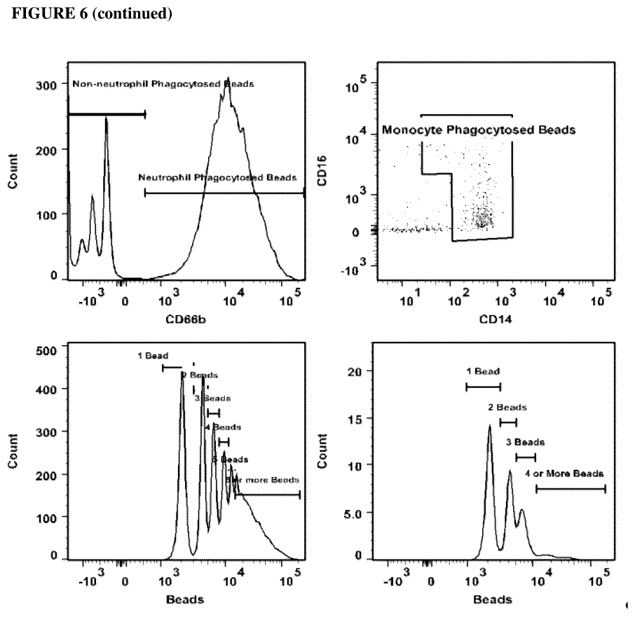

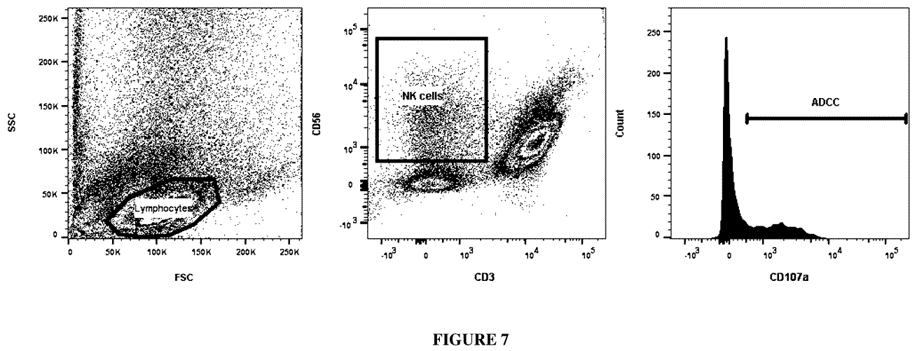

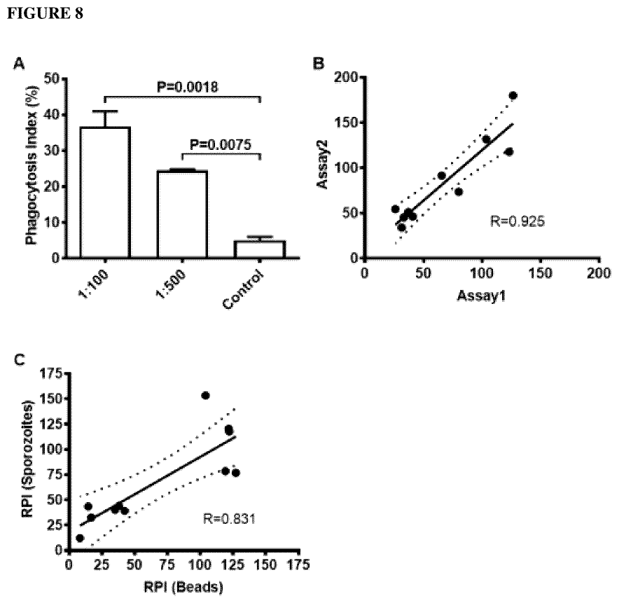

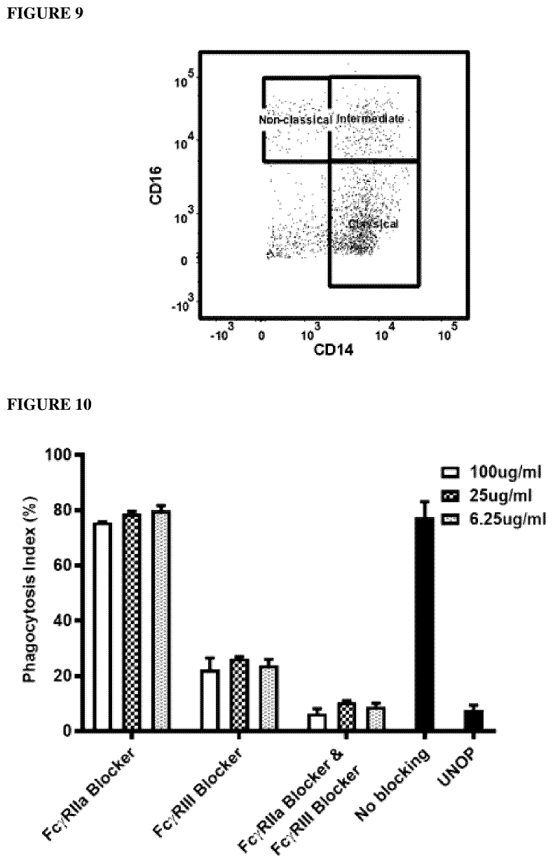

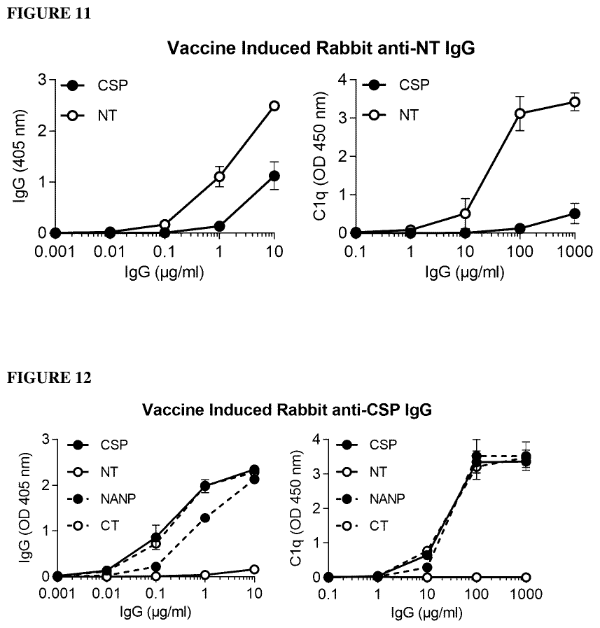

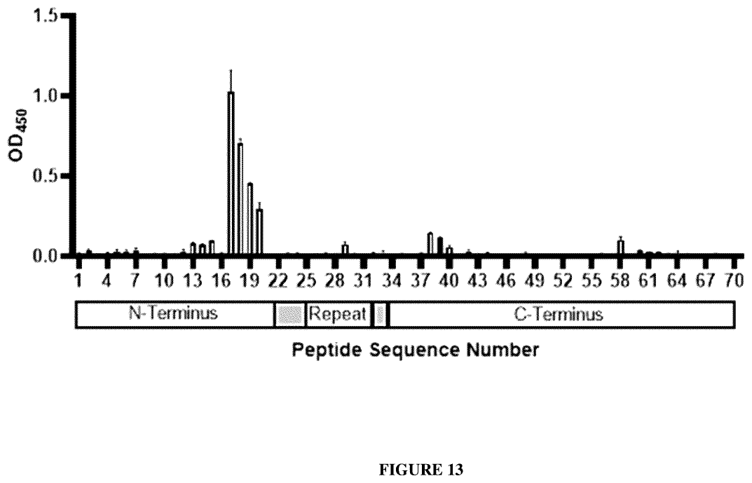

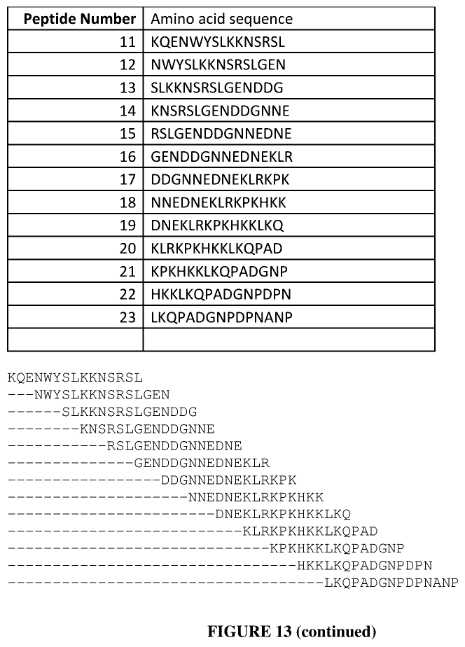

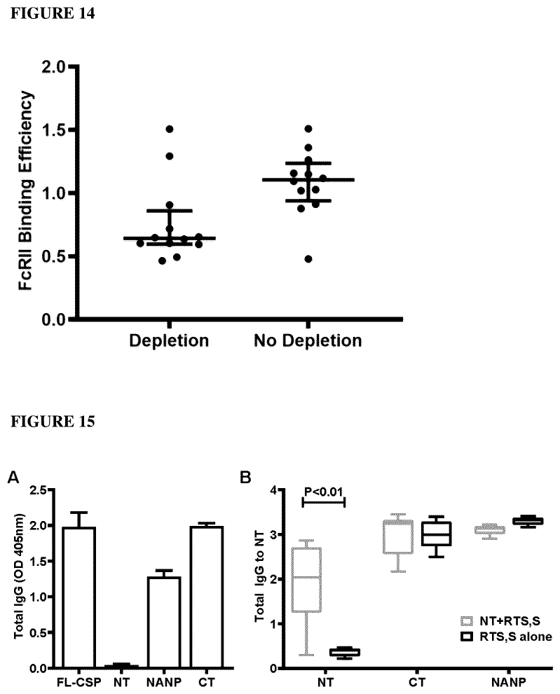

Cryo-preserved P. falciparum sporozoites opsonised with immune antibodies from immune adults were effectively phagocytosed by THP-1 cells in a concentration-dependent manner and there was little opsonic phagocytosis observed using antibodies from malaria-naïve donors (Melbourne residents) (FIG. 1A and legend). The magnitude of opsonic phagocytosis mediated by individual sera was highly reproducible (r=0.925, p<0.001; FIG. 1B and legend). Opsonic phagocytosis assays required large numbers of viable P. falciparum sporozoites, which are difficult to generate and limits the wider application of this assay to clinical studies. Therefore, we established the use of fluorescent beads coated with full-length CSP, the major antigen expressed on sporozoites, as a surrogate measure of opsonic phagocytosis of sporozoites. CSP-coated beads opsonised with a selection of antibody samples from different immune adults were effectively phagocytosed by THP-1 cells, and opsonic phagocytosis of CSP-coated beads was strongly correlated with opsonic phagocytosis of sporozoites (r=0.831, p<0.01, FIG. 1C and legend). Therefore, CSP-beads provided an efficient platform for studying antibody-mediated opsonic phagocytosis of sporozoites.

The cell types that mediate opsonic phagocytosis were investigated in a whole leukocyte assay with fresh blood, using CSP-beads opsonised with polyclonal rabbit antibody to CSP. Interestingly, the rate of phagocytosis (number of beads phagocytosed per cell) was much higher by neutrophils than that by monocytes (FIGS. 1A and B). Because the phagocytosis rate was quantified, the higher activity of neutrophils is not simply explained by their higher abundance compared to monocytes. Opsonic phagocytosis increased with increasing antibody concentrations, and the higher relative activity of neutrophils compared to monocytes was seen across high and low concentrations (FIG. 1A). In addition, neutrophils demonstrated higher phagocytic activity across a range of bead:cell ratios (FIG. 1B). The greater phagocytosis activity of neutrophils was confirmed using transgenic P. berghei sporozoites that express P. falciparum CSP (which replaces the endogenous P. berghei CSP gene (Triller et al 2017); referred to as PfCSP-P. berghei). Consistent with the previous observation using beads, phagocytosis of transgenic PfCSP-P. berghei sporozoites opsonised with a pool of sera from immune malaria-exposed donors was predominately mediated by neutrophils with only low levels of phagocytosis by monocytes, and neutrophils showing a higher phagocytosis rate (FIG. 1C). Further analysis of the monocyte population indicated that the majority of phagocytosis was by the classical subset (FIG. 1D; FIG. 1 and legend). Furthermore, neutrophils isolated from peripheral blood could effectively phagocytose transgenic PfCSP-P. berghei sporozoites that were opsonised with pooled serum antibodies from immune adults (FIG. 1E).

### Example 2—Defining Fcγ-Receptors Involved in Opsonic Phagocytosis of Sporozoites

To define the specific FcγRs involved in opsonic phagocytosis, we opsonised whole P. falciparum sporozoites and CSP-beads with pooled serum antibodies from immune adults and measured opsonic phagocytosis by neutrophils in the presence of specific FcγR-blocking antibodies. Neutrophils had substantially lower opsonic phagocytosis of both CSP-beads and P. falciparum sporozoites in the presence of either FcγRIIa or FcγRIII blockers (FIGS. 2A and B), suggesting that both receptors were important. Notably, FcγRIII blockade was the most effective at inhibiting phagocytosis of P. falciparum sporozoites or CSP-beads, with a weaker effect of FcγRIIa blockade. Opsonic phagocytosis by neutrophils was unaffected by blocking FcγRI. Titrating the concentration of blocking antibodies suggested we had achieved the maximal effect for blockade of each individual FcγR (See FIG. 2 and legend). Therefore, we combined FcγRIIa and FcγRIII blocking antibodies and found they gave greater inhibition of phagocytosis than using either blocking antibody on its own, further suggesting the importance of both FcγR types for optimal phagocytosis by neutrophils (FIG. 2B). These results are consistent with the expression of FcγRs on resting neutrophils, which predominantly express FcγRIIa and FcγRIIIa/b, but lack FcγRI. The importance of FcγRIII in mediating opsonic phagocytosis of CSP-beads and sporozoites may partly explain the relatively low activity of monocytes, since the major monocyte subsets express little FcγRIIIa on the surface in a non-activated state and do note express FcγRIIIb. THP-1 cells are widely used as a model of monocyte phagocytosis and highly express FcγRI, which is bound by monomeric IgG (whereas FcγRIIa and FcγRIII expressed by neutrophils only bind to immune complexes). Accordingly, we found that opsonic phagocytosis by THP-1 cells in standard conditions (containing 10% FCS only) was strongly inhibited by the FcγRI blocker with limited inhibition observed with the FcγRIIa blocker, and none with FcγRIII blocking (FIG. 2C). This suggested that the interaction between IgG and FcγRI was particularly important for THP-1 cells, contrasting results seen with neutrophils. Therefore, the standard THP-1 assay was modified to make it more physiologically-relevant by including human serum in the assay media. For opsonic phagocytosis of CSP-coated beads by THP-1 cells in the presence of additional 2.5% human serum (as used in the whole leukocyte and neutrophil assays), there was much less opsonic phagocytosis (FIG. 2D), presumably due to the binding of non-immune monomeric human IgG present in the human serum to FcγRI, and limited expression of FcγRIIIa. These findings may explain the low phagocytic activity of monocytes observed in the whole leukocyte assays in the presence of human serum.

Given the apparent importance of FcRγIII, the function of antibodies to CSP among immune adults was measured in ADCC assays, since FcRγIII interactions play a key role in mediating ADCC via NK cells. A selection of samples from the immune adult cohort (Kanyawegi cohort) was selected to represent high, intermediate and low responders (n=31) in an assay using an ADCC reporter cell line that only expresses FcγRIIIa. In this assay, naturally-acquired human antibodies among immune adults had significant activity whereas antibodies from Melbourne malaria-naïve donors did not induce ADCC (FIGS. 2E and F). These findings suggest that antibodies to CSP may also promote ADCC activity through engaging FcγRIIIa. Subsequently, we tested the activity of primary NK cells in fresh blood using an established assay (Lichtfuss et al, 2011). Incubation of NK cells with CSP-beads opsonised by the selection of immune adults lead to degranulation of primary NK cells (FIGS. 2G and H). The activity varied across samples tested. Interestingly, primary NK cell degranulation activity significantly correlated with FcγRIII binding (r=0.538, p=0.002; FIG. 2H), but weakly correlated with IgG to CSP (r=0.127, P=0.489, n=31).

### Example 3—Direct Binding of FcγRIII and FcγRIIa by Antibody-Antigen Complexes

To further understand the importance of FcγR-antibody binding in immunity to sporozoites, FcγR-binding assays were developed using recombinant FcγRIIa and FcγRIII that were expressed and purified as dimers so they can interact with antigen-antibody complexes (see Wines et al 2016). Immune adults had high prevalence and magnitude of antibodies to CSP that could interact with FcγRIIa and FcγRIII dimers (representative examples shown in FIG. 3A). Overall 67.3% and 61.5% of samples (n=104) were considered positive for FcγRIIa and FcγRIII binding. While the binding of both FcγRs had a strong, positive correlation among samples (r=0.71, p<0.0001, n=104) (FIG. 3B), there was greater binding to FcγRIII than to FcγRIIa overall, which is consistent with the earlier data suggesting that FcγRIII may play a more prominent role in phagocytosis (FIGS. 2A and B). Additionally, some individual samples demonstrated high binding to one FcγR and low binding to the other. Furthermore, the ability of antibodies to CSP to promote direct binding of FcγRIII was correlated with ADCC tested by both the ADCC reporter cell line (r=0.405, P=0.022, FIG. 2F) and primary NK cells (r==0.538, P=0.002, FIG. 2H).

### Example 4—Regions of CSP Targeted by Functional Antibodies

The regions of CSP that were targeted by functional antibodies was investigated. Rabbit anti-CSP antibodies were previously shown to promote opsonic phagocytosis of CSP-beads, and their functional activity against whole sporozoites was confirmed herein. P. falciparum sporozoites opsonised with these antibodies resulted in a high phagocytosis index (40.5%) compared to control (FIG. 4A). Further, a mouse monoclonal antibody (mAb) 2H8 (IgG2a subclass) against the central NANP-repeat region of P. falciparum CSP (Kurtovic et al, 2018) promoted substantial phagocytosis (17.4%, FIG. 4B), that was greater than seen with the negative control mAb 3D11 that targets CSP of P. berghei which does not have the NANP-repeat epitope (FIG. 4B).

It was determined whether different regions of CSP were targets of opsonic phagocytosis using three protein constructs; i) a long synthetic peptide representing the NT region (which is predicted to be unstructured, and not included in the RTS,S vaccine), ii) a synthetic peptide representing the repeat region (NANP repeats), and iii) a recombinant protein representing the CT region. Each construct was separately coated onto fluorescent beads (at saturating concentrations) and opsonised with the pool of immune sera. Antibodies effectively promoted neutrophil phagocytosis of beads coated with all three constructs, with the NT region showing the highest levels of opsonic phagocytosis (FIG. 4C). To further define the role of antibodies to the CT and NT regions, rabbit polyclonal antibodies were generated to each region, and these antibodies were used to opsonize transgenic PfCSP-P.berghei sporozoites for phagocytosis by isolated neutrophils. Rabbit polyclonal antibodies to both the CT and NT regions effectively promoted opsonic phagocytosis of PfCSP-P.berghei sporozoites (FIG. 4D). Since the concentration of IgG to the CT and NT from vaccinated rabbit sera differed, phagocytosis relative to IgG levels was assessed (calculated as phagocytosis activity divided by IgG reactivity determined by ELISA). This showed that antibodies to the N-terminal region had substantially higher relative phagocytic activity compared to CT antibodies.

Regions of CSP (NT region, central repeat region and CT region) were assessed that were targets of naturally-acquired antibodies for their ability to promote engagement of FcγRIIa and FcγRIII with antibodies from the immune adult serum pool. The results established that acquired human antibodies opsonised each CSP region with sufficient density to promote binding of FcγRIIa and FcγRIII (FIG. 4E). As the total level of IgG binding varied among different CSP constructs FcγRIIa and FcγRIII binding was standardised relative to the level of IgG reactivity (termed FcγR binding efficiency). This suggested that antibodies to the NT region had a higher potential to engage FcγRs than the central repeat and CT regions.

### Example 5—NT Region Able to Elicit IgG Antibodies and Complement Fixation Activity

Antibodies raised against the NT region were able to promote complement fixation. Prior studies have demonstrated that complement fixation can lead to sporozoite killing and inhibition of sporozoite motility and invasion. Rabbit antibodies raised against the NT region of CSP were tested for IgG reactivity (left panel) and complement fixation activity (C1q binding; right panel) using immobilised full-length CSP (lower) or N-terminal protein (upper). Results (see FIG. 11) show mean and range from two independent experiments.

### Example 6—Vaccination with Full Length CSP does not Generate Antibodies to N-Terminal Region

Immunization with full-length CSP that includes the N-terminal region does not generate good antibodies to the N-terminal region. Instead, as determined herein, specific constructs are needed to achieve this. Vaccination of rabbits with full-length CSP does not effectively generate antibodies to the N-terminal region of CSP (FIG. 12—bottom pink line). Data show IgG reactivity (left panel) of antibodies to different regions of CSP (NT, NANP, and CT regions; full-length CSP for comparison) and complement fixation activity (C1q binding; right panel). Mean and range of two independent experiments.

### Example 7—Specific Overlapping Peptides Derived from N-Terminal with Polyclonal IgG

Since antibodies to the NT regions were highly effective in activating FcγRs and promoting opsonic phagocytosis, and in some embodiments also promote complement fixation, epitope mapping was performed of the rabbit polyclonal antibodies against the NT region using an overlapping peptide array. Antibodies were reactive with peptides covering a region that is close to the junction between the N-terminal region and the NANP/NVPD repeat region, but did not include the recently reported junctional epitope (Tan et al. 2018 Nature Medicine published online 19 Mar. 2018; doi:10.1038/nm.4513), which sits between the N-terminal region and NANP repeat region.

Antibodies were tested against an overlapping peptide array derived from the CSP sequence (3D7 strain). N-terminal peptide sequences are listed below. Bars and error bars represents mean and standard error from two independent experiments.

Peptides 13, 14 and 15 together with peptides 17, 18, 19 and 20 were interactive with IgG raised against the N-terminus of CSP. FIG. 13 illustrates the results of epitope mapping using polyclonal rabbit IgG raised against the N-terminus of CSP. There is some cross reactivity with peptides within the C-terminus, but this is at very low levels and would not be expected to contribute to the functional activity of the antibodies. The antibodies to the NT region showed very little reactivity to recombinant protein representing the whole C-terminal region (FIG. 5). Rabbit antibodies generated against the NT region were tested for reactivity against an array of overlapping peptides (15 aa with 12 aa overlap) including the NT region, NANP/NVDP repeat and the junctional region. Data show IgG reactivity to different peptides; the reactive peptides include a 21-aa region corresponding to CSP residues 76-96 (P=0.036 for differences in peptide reactivity, Kruskal-Wallis test, two experiments). Error bars represent the range from two experimental repeats.

### Example 8—IgG Antibodies to N-Terminus Promote FcγRIIa Binding

Serum antibodies (from different malaria-exposed adults) were incubated with recombinant NT protein to deplete NT antibodies from the pool. The ability of antibodies to promote FcRIIa binding was then tested with/without NT-antibody depletion and further standardized according to the total IgG titre to full-length CSP with/without depletion (FcγRIIa binding efficiency). The FcγRIIa binding efficiency was significantly reduced after the NT specific antibodies were depleted (P=0.021). FIG. 14 illustrates how depletion of antibodies to the N-terminal region of CSP among human antibodies decreased FcγRIIa binding efficiency of the antibodies.

Further experiments to determine one or more epitope that stimulate antibodies that promote sporozoite phagocytosis or complement mediated functions are undertaken.

### Example 9—Vaccination with a Combination of RTS,S and CSP N-Terminal Peptide Elicits Effective IgG to all Three Regions of CSP (NT, CT and NANP)

Mice were vaccinated with a mixture of CSP antigens/peptides corresponding to the RTS,S vaccine construct sequence either on its own (RTS,S alone), or mixed with a synthetic peptide sequence from the N-terminal region of CSP (represented in FIG. 15 B as NT+RTS,S). After vaccination, mouse serum was tested for the presence of IgG to the N-terminal (NT), C-terminal (CT), and NANP-repeat regions of CSP. Results (See FIG. 15) demonstrate that a mixture of the RTS,S immunogen and the NT peptide immunogen effectively generates IgG to all 3 regions of CSP. Thus by generating functional antibodies to the CSP NT region the protective effect of malaria vaccines is enhanced.

Vaccines were formulated with Quil-A adjuvant, and 3 doses were given:
Group 1: 5 μg RTS,S immunogen per dose
Group 2: 5 μg RTS,S immunogen+10 μg NT peptide.

The RTS,S construct includes the CT and NANP regions, but does not include the NT region. The NT peptide used was based on the following sequence:ENWYSLKKNSRSLGENDDGNNEDNEKLRKPKHKKLKQPADG (SEQ ID NO: 57). The peptide was fused to a universal T-cell helper epitope derived from tetanus toxoid—QYIKANSKFIGITEL (SEQ ID NO: 58)—with a spacer (SGSG)(SEQ ID NO: 59) between the NT sequence and the T-cell epitope. The complete NT-peptide sequence for vaccination was:

(SEQ ID NO: 60)
ENWYSLKKNSRSLGENDDGNNEDNEKLRKPKHKKLKQPADSGSGQYIK
ANSKFIGITEL.

Current leading vaccines, RTS,S (GSK) and R21 (Jenner Institute, Oxford) are based on a CSP sequence that includes the C-terminal region (CT) and central repeat sequence (NANP), but do not include the N-terminal (NT) sequence of CSP. Vaccination with full-length CSP is not effective in generating antibodies to the N-terminal region of CSP. Vaccination of mice with a mixture of the RTS,S immunogen and a immunogen based on part of the sequence of the N-terminal region effectively generated IgG to all major regions of CSP (C-terminal, central repeat, and N-terminal regions). The peptide was fused to a universal T-cell helper epitope derived from tetanus toxoid—QYIKANSKFIGITEL. These findings show that adding a N-terminal peptide immunogen as a mixture with other CSP-based vaccines is an effective strategy to generate antibodies to the N-terminal region of CSP, as well as other regions of CSP. Vaccination with full-length CSP is not an effective strategy.

TABLE 1
Experimental materials and settings of opsonic phagocytosis assays

| | | | Specific | |
|---|---|---|---|---|
| | | | experiment | |
| Targets | Effector cell | Opsonin | condition | Aim of experiment |
| P. falciparum | THP-1 cells | Malaria | Serum free | Determine whether |
| sporozoites | | exposed | | THP-1 cells can |
| | | adult sera | | phagocytose antibody |
| | | | | opsonised sporozoites |
| P. falciparum | Neutrophils | Malaria | Neutrophils were | Determine whether |
| sporozoites | | exposed | used in 2.5% | neutrophils can |
| | | adult sera | human serum | phagocytose antibodies |
| | | | media | opsonised sporozoites |
| P. falciparum | Neutrophils | CSP | Neutrophils were | Is CSP is a major |
| sporozoites | | specific | used in 2.5% | antibody target for |
| | | rabbit IgG | human serum | promoting opsonic |
| | | | media | phagocytosis? |
| P. berghei | Neutrophils | Malaria | Neutrophils were | Is CSP is a major target |
| sporozoites | | exposed | used in 2.5% | of naturally-acquired |
| expressing PfCSP | | adult sera | human serum | antibodies that promote |
| | | | media | opsonic phagocytosis |
| P. berghei | Whole | Malaria | Leukocytes were | Confirming that CSP is a |
| sporozoites | leukocytes | exposed | used in 2.5% | major target of natural |
| expressing PfCSP | | adult sera | human serum | acquired antibody to |
| or Beads coated | | or CSP | media | promote opsonic |
| with PfCSP | | specific | | phagocytosis and |
| | | rabbit IgG | | confirming neutrophils |
| | | | | are the key immune |
| | | | | cells mediating opsonic |
| | | | | phagocytosis of |
| | | | | sporozoites |
| Beads coated | Neutrophils | Malaria | Neutrophils were | To estimate the level of |
| with PfCSP or | | exposed | used in 2.5% | antibody mediated |
| constructs | | adult sera | human serum | opsonic phagocytosis in |
| representing | | | media | populations |
| PfCSP regions | | | | |
| P. falciparum | THP-1 cells | Malaria | THP-1 cells and | To determine the role of |
| sporozoites or | or | exposed | the neutrophils | each FcγR in opsonic |
| beads coated | neutrophils | adult sera | were treated | phagocytosis of |
| with CSP | | | with different | sporozoites by |
| | | | FcγR blockers | neutrophils and THP-1 |
| | | | | cells |
| P. falciparum | Neutrophils | Malaria | Neutrophils were | To determine the |
| merozoites | | exposed | used in 2.5% | acquisition of antibodies |
| | | adult sera | human serum | that promote |
| | | | media | phagocytosis of |
| | | | | merozoites by |
| | | | | neutrophils |

TABLE 2
Amino acid sub-classification

| Sub-classes | Amino acids |
|---|---|
| Acidic | Aspartic acid, Glutamic acid |
| Basic | Noncyclic: Arginine, Lysine; Cyclic: Histidine |
| Charged | Aspartic acid, Glutamic acid, |
| | Arginine, Lysine, Histidine |
| Small | Glycine, Serine, Alanine, Threonine, Proline |
| Polar/neutral | Asparagine, Histidine, Glutamine, |
| | Cysteine, Serine, Threonine |
| Polar/large | Asparagine, Glutamine |
| Hydrophobic | Tyrosine, Valine, Isoleucine, Leucine, |
| | Methionine, Phenylalanine, Tryptophan |
| Aromatic | Tryptophan, Tyrosine, Phenylalanine |
| Residues that influence | Glycine and Proline |
| chain orientation | |

TABLE 3
Exemplary and Preferred Amino Acid Substitutions

| Original | Exemplary | Preferred |
|---|---|---|
| residue | substitutions | substitutions |
| Ala | Val, Leu, Ile | Val |
| Arg | Lys, Gln, Asn | Lys |
| Asn | Gln, His, Lys, Arg | Gln |
| Asp | Glu | Glu |
| Cys | Ser | Ser |
| Gln | Asn, His, Lys, | Asn |
| Glu | Asp, Lys | Asp |
| Gly | Pro | Pro |
| His | Asn, Gln, Lys, Arg | Arg |
| Ile | Leu, Val, Met, Ala, Phe, Norleu | Leu |
| Leu | Norleu, Ile, Val, Met, Ala, Phe | Ile |
| Lys | Arg, Gln, Asn | Arg |
| Met | Leu, Ile, Phe | Leu |
| Phe | Leu, Val, Ile, Ala | Leu |
| Pro | Gly | Gly |
| Ser | Thr | Thr |
| Thr | Ser | Ser |
| Trp | Tyr | Tyr |
| Tyr | Trp, Phe, Thr, Ser | Phe |
| Val | Ile, Leu, Met, Phe, Ala, Norleu | Leu |

KEY TO SEQUENCE LISTING

| SEQ ID NO: | DESCRIPTION | SEQUENCE |
|---|---|---|
| SEQ ID NO: 1 | N-terminal amino | KQENWYSLKKNSRSLGENDDGNNED |
| | acids 58 to 104 of | NEKLRKPKHKKLKQPADGNPDP |
| | CSP | |
| SEQ ID NO: 2 | Amino acids 58 to 81 | KQENWYSLKKNSRSLGENDDGNNE |
| SEQ ID NO: 3 | Amino acids 64 to 84 | SLKKNSRSLGENDDGNNEDNE |
| SEQ ID NO: 4 | Amino acids 82 to 104 | DNEKLRKPKHKKLKQPADGNPDP |
| SEQ ID NO: 5 | Amino acids 76 to 100 | DDGNNEDNEKLRKPKHKKLKQPADG |
| SEQ ID NO: 6 | | DDG |
| SEQ ID NO: 7 | | GNN |
| SEQ ID NO: 8 | | DDGN |
| SEQ ID NO: 9 | | DDGNN |
| SEQ ID NO: 10 | | DDGNNE |
| SEQ ID NO: 11 | | DDGNNED |
| SEQ ID NO: 12 | | DDGNNEDN |
| SEQ ID NO: 13 | | DDGNNEDNE |
| SEQ ID NO: 14 | | DDGNNEDNEK |
| SEQ ID NO: 15 | | DDGNNEDNEKL |
| SEQ ID NO: 16 | | DDGNNEDNEKLR |
| SEQ ID NO: 17 | | DDGNNEDNEKLRK |
| SEQ ID NO: 18 | | DDGNNEDNEKLRKP |
| SEQ ID NO: 19 | | DDGNNEDNEKLRKPK |
| SEQ ID NO: 20 | | DDGNNEDNEKLRKPKH |
| SEQ ID NO: 21 | | DDGNNEDNEKLRKPKHK |
| SEQ ID NO: 22 | | DDGNNEDNEKLRKPKHKK |
| SEQ ID NO: 23 | | DDGNNEDNEKLRKPKHKKL |
| SEQ ID NO: 24 | | DDGNNEDNEKLRKPKHKKLK |
| SEQ ID NO: 25 | | DDGNNEDNEKLRKPKHKKLKQ |
| SEQ ID NO: 26 | | DDGNNEDNEKLRKPKHKKLKQP |
| SEQ ID NO: 27 | | DDGNNEDNEKLRKPKHKKLKQPA |
| SEQ ID NO: 28 | | DDGNNEDNEKLRKPKHKKLKQPAD |
| SEQ ID NO: 29 | | KQENWYSLKKNSRSLGENDDGNN |
| SEQ ID NO: 30 | | QENWYSLKKNSRSLGENDDGNN |
| SEQ ID NO: 31 | | KNSRSLGENDDGNN |
| SEQ ID NO: 32 | | NSRSLGENDDGNN |
| SEQ ID NO: 33 | | RSLGENDDGNN |
| SEQ ID NO: 34 | | SLGENDDGNN |
| SEQ ID NO: 35 | | LGENDDGNN |
| SEQ ID NO: 36 | | GENDDGNN |
| SEQ ID NO: 37 | | KQENWYSLKKNSRSLGENDDGNN |
| SEQ ID NO: 38 | | ENDDGNN |
| SEQ ID NO: 39 | | NDDGNN |
| SEQ ID NO: 40 | | DDG |
| SEQ ID NO: 41 | Amino acids 59 to 327 | QENWYSLKKNSRSLGENDDGNNEDN |
| | of CSP | EKLRKPKHKKLKQPADGNPDPNANP |
| | | NVDPNANPNVDPNANPNVDPNANPN |
| | | ANPNANPNANPNANPNANPNANPNA |
| | | NPNANPNANPNANPNANPNANPNAN |
| | | PNANPNANPNANPNVDPNANPNANP |
| | | NANPNANPNANPNANPNANPNANPN |
| | | ANPNANPNANPNANPNANPNANPNA |
| | | NPNANPNANPNANPNKNNQGNGQGH |
| | | NMPNDPNRNVDENANANSAVKNNN |
| | | NEEPSDKHIKEYLNKIQNSLSTEWSPC |
| | | SVTCGNGIQVRIKPGSANKPKDELDY |
| | | ANDIEKKICKMEKCSSVFNVVNSGS |
| SEQ ID NO: 42 | Peptide 11 to 23 in | KQENWYSLKKNSRSL |
| | FIG. 13 | |
| SEQ ID NO: 43 | Peptide 12 | NWYSLKKNSRSLGEN |
| SEQ ID NO: 44 | Peptide 13 | SLKKNSRSLGENDDG |
| SEQ ID NO: 45 | Peptide 14 | KNSRSLGENDDGNNE |
| SEQ ID NO: 46 | Peptide 15 | RSLGENDDGNNEDNE |
| SEQ ID NO: 47 | Peptide 16 | GENDDGNNEDNEKLR |
| SEQ ID NO: 48 | Peptide 17 | DDGNNEDNEKLRKPK |
| SEQ ID NO: 49 | Peptide 18 | NNEDNEKLRKPKHKK |
| SEQ ID NO: 50 | Peptide 19 | DNEKLRKPKHKKLKQ |
| SEQ ID NO: 51 | Peptide 20 | KLRKPKHKKLKQPAD |
| SEQ ID NO: 52 | Peptide 21 | KPKHKKLKQPADGNP |
| SEQ ID NO: 53 | Peptide 22 | HKKLKQPADGNPDPN |
| SEQ ID NO: 54 | Peptide 23 | LKQPADGNPDPNANP |
| SEQ ID NO: 55 | Amino acid sequence | MMRKLAILSVSSFLFVEALFQEYQCY |
| | of CSP 3D7 | GSSSNTRVLNELNYDNAGTNLYNELE |
| | | MNYYGKQENWYSLKKNSRSLGENDD |
| | | GNNEDNEKLRKPKHKKLKQPADGNP |
| | | DPNANPNVDPNANPNVDPNANPNVD |
| | | PNANPNANPNANPNANPNANPNANP |
| | | NANPNANPNANPNANPNANPNANPN |
| | | ANPNANPNANPNANPNANPNVDPNA |
| | | NPNANPNANPNANPNANPNANPNAN |
| | | PNANPNANPNANPNANPNANPNANP |
| | | NANPNANPNANPNANPNANPNKNNQ |
| | | GNGQGHNMPNDPNRNVDENANANS |
| | | AVKNNNNEEPSDKHIKEYLNKIQNSLS |
| | | TEWSPCSVTCGNGIQVRIKPGSANKPK |
| | | DELDYANDIEKKICKMEKCSSVFNVV |
| | | NSSIGLIMVLSFLFLN |
| SEQ ID NO: 56 | Nucleic acid sequence | ATGATGAGAAAATTAGCTATTTTAT |
| | encoding CSP 3D7 | CTGTTTCTTCCTTTTTATTTGTTGAGG |
| | | CCTTATTCCAGGAATACCAGTGCTAT |
| | | GGAAGTTCGTCAAACACAAGGGTTC |
| | | TAAATGAATTAAATTATGATAATGC |
| | | AGGCACTAATTTATATAATGAATTA |
| | | GAAATGAATTATTATGGGAAACAGG |
| | | AAAATTGGTATAGTCTTAAAAAAAA |
| | | TAGTAGATCACTTGGAGAAAATGAT |
| | | GATGGAAATAACGAAGACAACGAG |
| | | AAATTAAGGAAACCAAAACATAAA |
| | | AAATTAAAGCAACCAGCGGATGGTA |
| | | ATCCTGATCCAAATGCAAACCCAAA |
| | | TGTAGATCCCAATGCCAACCCAAAT |
| | | GTAGATCCAAATGCAAACCCAAATG |
| | | TAGATCCAAATGCAAACCCAAATG |
| | | CAAACCCAAATGCAAACCCAAATGC |
| | | AAACCCAAATGCAAACCCAAATGCA |
| | | AACCCAAATGCAAACCCAAATGCAA |
| | | ACCCAAATGCAAACCCAAATGCAAA |
| | | CCCAAATGCAAACCCAAATGCAAAC |
| | | CCAAATGCAAACCCAAATGCAAACC |
| | | CCAATGCAAATCCTAATGCAAACCC |
| | | AAATGCAAACCCAAACGTAGATCCT |
| | | AATGCAAATCCAAATGCAAACCCAA |
| | | ACGCAAACCCCAATGCAAATCCTAA |
| | | TGCAAACCCCAATGCAAATCCTAAT |
| | | GCAAATCCTAATGCCAATCCAAATG |
| | | CAAATCCAAATGCAAACCCAAACGC |
| | | AAACCCCAATGCAAATCCTAATGCC |
| | | AATCCAAATGCAAATCCAAATGCAA |
| | | ACCCAAATGCAAACCCAAATGCAAA |
| | | CCCCAATGCAAATCCTAATAAAAAC |
| | | AATCAAGGTAATGGACAAGGTCACA |
| | | ATATGCCAAATGACCCAAACCGAAA |
| | | TGTAGATGAAAATGCTAATGCCAAC |
| | | AGTGCTGTAAAAAATAATAATAACG |
| | | AAGAACCAAGTGATAAGCACATAAA |
| | | AGAATATTTAAACAAAATACAAAAT |
| | | TCTCTTTCAACTGAATGGTCCCCATG |
| | | TAGTGTAACTTGTGGAAATGGTATT |
| | | CAAGTTAGAATAAAGCCTGGCTCTG |
| | | CTAATAAACCTAAAGACGAATTAGA |
| | | TTATGCAAATGATATTGAAAAAAAA |
| | | ATTTGTAAAATGGAAAAATGTTCCA |
| | | GTGTGTTTAATGTCGTAAATAGTTCA |
| | | ATAGGATTAATAATGGTATTATCCTT |
| | | CTTGTTCCTTAATTAGG |
| SEQ ID NO: 57 | PCMS or N-terminal | ENWYSLKKNSRSLGENDDGNNEDNE |
| | CSP antigen or | KLRKPKHKKLKQPADG |
| | peptide amino acid | |
| | sequence | |
| SEQ ID NO: 58 | T-cell helper epitope | QYIKANSKFIGITEL |
| | from tetanus toxoid | |
| SEQ ID NO: 59 | spacer | SGSG |
| SEQ ID NO: 60 | PCMS/N-terminal | ENWYSLKKNSRSLGENDDGNNEDNE |
| | CSP peptide amino | KLRKPKHKKLKQPADSGSGQYIKANS |
| | acid sequence linked | KFIGITEL |
| | via a spacer to a T-cell | |
| | helper epitope | |

All documents cited or referenced herein, and all documents cited or referenced in herein cited documents, together with any manufacturer's instructions, descriptions, product specifications, and product sheets for any products mentioned herein or in any document incorporated by reference herein, are hereby incorporated herein by reference in their entirety

Those of skill in the art will appreciate that, in light of the instant disclosure, various modifications and changes can be made in the particular embodiments exemplified without departing from the scope of the present invention. All such modifications and changes are intended to be included within the scope of the appended claims.

### BIBLIOGRAPHY

Beeson, J. G., et al. Challenges and strategies for developing efficacious and long-lasting malaria vaccines, Sci Transl Med 11 (2019).
Dobano, C., et al. Concentration and avidity of antibodies to different circumsporozoite epitopes correlate with RTS,S/AS01E malaria vaccine efficacy. Nat Commun 10, 2174 (2019).
Triller, G., et al. Natural Parasite Exposure Induces Protective Human Anti-Malarial Antibodies. Immunity 47, 1197-1209 e1110 (2017).
Irani, V., et al. Molecular properties of human IgG subclasses and their implications for designing therapeutic monoclonal antibodies against infectious diseases. Molecular immunology (2015).
Kolaczkowska, E. & Kubes, P. Neutrophil recruitment and function in health and inflammation. Nat Rev Immunol 13, 159-175 (2013).
Gordon, S. Phagocytosis: An Immunobiologic Process. Immunity 44, 463-475 (2016).
Golay, J., et al. Human neutrophils express low levels of FcgammaRIIIA, which plays a role in PMN activation. Blood 133, 1395-1405 (2019).
Quinn, M. T., DeLeo, F. R. & Bokoch, G. M. Neutrophil methods and protocols. in Methods Mol Biol, Vol. 412 (ed. Quinn, M. T.) 13-18 (Springer Science+Business Media, Springer New York Heidelberg Dordrecht London, 2007).
Osier, F. H., et al. Opsonic phagocytosis of Plasmodium falciparum merozoites: mechanism in human immunity and a correlate of protection against malaria. BMC Med 12, 108 (2014).
Kurtovic, L., et al. Human antibodies activate complement against Plasmodium falciparum sporozoites, and are associated with protection against malaria in children. BMC Med 16, 61 (2018).
Kurtovic et al Frontiers in Immunology Vol. 12, Article 641421 March 2021.
Wines, B. D., et al. Dimeric FcgammaR Ectodomains as Probes of the Fc Receptor Function of Anti-Influenza Virus IgG. J Immunol 197, 1507-1516 (2016).
Lichtfuss, G. F., et al. HIV inhibits early signal transduction events triggered by CD16 cross-linking on N K cells, which are important for antibody-dependent cellular cytotoxicity. J Leukoc Biol 89, 149-158 (2011).
Steel, R. W., et al. An Opsonic Phagocytosis Assay for Plasmodium falciparum Sporozoites. Clin Vaccine Immunol 24 (2017).
Kester, K. E., et al. Randomized, double-blind, phase 2a trial of falciparum malaria vaccines RTS,S/AS01B and RTS,S/AS02A in malaria-naive adults: safety, efficacy, and immunologic associates of protection. J Infect Dis 200, 337-346 (2009).
Deveraux et al. Nucleic Acids Research 12: 387-395 (1984).
Roberge et al. Science 269: 202 (1995).
Tan et al. Nature Medicine published online 19 Mar. 2018; doi:10.1038/nm.4513 (2018).

1. A method of inducing in a subject antibodies against P. falciparum (Pf) parasites/sporozoites for vaccinating the subject, comprising administering to the subject one or more peptide antigens representing specific subregions of a circumsporozoite polypeptide (CSP) selected from NT, and CT and/or NANP subregions, wherein the method comprises:
(i) administering a peptide antigen or a sequence encoding a peptide antigen presenting N-terminal (NT) epitopes of PfCSP and not representing NANP or CT subregions of PfCSP; or
(ii) co-administering a peptide antigen or sequence of (i) and a peptide or a sequence encoding a peptide presenting NANP and/or CT epitopes of PfCSP; or
(iii) administering a peptide or a sequence encoding a peptide representing NT, and CT and/or NANP subregions of PfCSP but not full length-PfCSP PfCSP,
wherein the NT subregion of CSP is amino acids 58 to 104 (SEQ ID NO: 1) of the P. falciparum CSP and the peptide presenting NT epitopes of SEQ ID NO: 1 comprises 3 to 48 contiguous amino acids from the N-terminal amino acids 58 to 104 (SEQ ID NO: 1) of CSP of strain 3D7, or a corresponding peptide from a different P. falciparum strain.

2. The method of claim 1, wherein the NT subregion of CSP is amino acids 58 to 81 (SEQ ID NO: 2) of the P. falciparum CSP and the peptide presenting NT epitopes of SEQ ID NO: 2 comprises 3 to 24 contiguous amino acids of SEQ ID NO: 2 of strain 3D7 or a corresponding peptide from a different P. falciparum strain.

3. The method of claim 1, wherein the NT subregion is amino acids 64 to 84 (SEQ ID NO: 3) of the PfCSP and the peptide presenting NT epitopes of SEQ ID NO: 3 comprises 3 to 21 contiguous amino acids of SEQ ID NO: 3 or a corresponding peptide from a different strain.

4. The method of claim 1, wherein the peptide representing NT epitopes comprises 3 to 24 contiguous amino acids from the N-terminal amino acids 58 to 81 (SEQ ID NO: 2) of CSP or a corresponding sequence from a different strain, and additionally comprises 3 to 24 contiguous amino acids from the N-terminal amino acids 82 to 104 (SEQ ID NO: 4) of CSP or a corresponding peptide from a different strain.

5. The method of claim 1, wherein one of the following applies:
(i) the peptide presenting CSP NT epitopes comprises 3 to 21 contiguous amino acids from the N-terminal amino acids 64 to 84 of the CSP polypeptide wherein at least 3 contiguous amino acids include DDG or GNN, and wherein the peptide and additionally comprises 3 to 24 contiguous amino acids from the N-terminal amino acids 76 to 100 (SEQ ID NO: 5) of the CSP polypeptide wherein at least 3 contiguous amino acids include KPK and not GNP;
(ii) the peptide presenting NT epitopes of SEQ ID NO: 1 comprises 9 to 48 contiguous amino acids from the N-terminal amino acids 58 to 104 (SEQ ID NO: 1);
(iii) the peptide presenting NT epitopes of SEQ ID NO: 1 comprises 12 to 48 contiguous amino acids from the N-terminal amino acids 58 to 104 (SEQ ID NO: 1); and
(iv) the peptide presenting NT epitopes of SEQ ID NO: 1 comprises 15 to 48 contiguous amino acids from the N-terminal amino acids 58 to 104 (SEQ ID NO: 1).

6. The method of claim 1, wherein the peptide presenting CSP NT epitopes comprises an amino acid sequence selected from one or more of SEQ ID NO: 1 to SEQ ID NO: 39, or SEQ ID NO: 57.

7. The method of claim 1, wherein the peptide presenting CSP NT epitopes comprises a T-cell helper epitope.

8. The method of claim 1, wherein the peptide representing CSP NT epitopes comprises a heterologous T-cell helper epitope.

9. The method of claim 1, wherein the peptide presenting CSP NANP or CT epitopes of PfCSP is RTS,S or R21 vaccine peptide.

10. The method of claim 1, wherein one of the following applies:
(i) the peptide representing NT, CT and NANP subregions of PfCSP comprises amino acids 59 to 327 or 60 to 327 of CSP (SEQ ID. NO:6) QENWYSLKKNSRSLGENDDGNNEDNEKLRKPKHKKLKQPADGNPDPNANPNVDPNANPNVDPNANPNVDPNAN PNANPNANPNANPNANPNANPNANPNANPNANPNANPNANPNANPNANPNANPNANPNANPNANPNVDPN ANPNANPNANPNANPNANPNANPNANPNANPNANPNANPNANPNANPNANPNANPNANPNANPNANPNAN PNKNNQGNGQGHNMPNDPNRNVDENANANSAVKNNNNEEPSDKHIKEYLNKIQNSLSTEWSPCSVTCGNGIQVR IKPGSANKPKDELDYANDIEKKICKMEKCSSVFNVVNSGS, or a corresponding sequence from a different Pf strain;
(ii) the peptide presenting CSP NT epitopes comprises an amino acid sequence of SEQ ID NO: 13;
(iii) the peptide presenting CSP NT epitopes comprises an amino acid sequence of SEQ ID NO: 23; and
(iv) the peptide presenting CSP NT epitopes comprises an amino acid sequence of SEQ ID NO: 24

11. The method of claim 1, wherein the peptide presenting NT epitopes comprises DDGNNEDNEKLRKPKHKKLKQ (SEQ ID NO: 25), ENWYSLKKNSRSLGENDDGNNEDNEKLRKPKHKKLKQPADG (SEQ ID NO: 57) or ENWYSLKKNSRSLGENDDGNNEDNEKLRKPKHKKLKQPADSGSGQYIKANSKFIGITEL (SEQ ID NO: 60.

12. The method of claim 1, wherein the antigen/s are administered in protein and/or nucleic acid form together with nanocarriers/liposomes, viral vectors, and/or in pharmaceutical compositions comprising an adjuvant or immunomodulatory agent.

13. A pharmaceutical composition comprising a peptide antigen or antigen encoding sequence representing the NT subregion of CSP of SEQ ID NO: 1, wherein the antigen comprises a peptide that presents NT epitopes of SEQ ID NO: 1 when administered to a subject and comprises 3 to 48 contiguous amino acids of SEQ ID NO: 1, or a corresponding peptide from a variant P. falciparum strain, and a pharmaceutically acceptable excipient or diluent.

14.-15. (canceled)

16. The composition of claim 13, wherein the CSP NT peptide comprises DDGNNEDNEKLRKPKHKKLKQ (SEQ ID NO: 25) or ENWYSLKKNSRSLGENDDGNNEDNEKLRKPKHKKLKQPADG (SEQ ID NO: 57) or KQENWYSLKKNSRSLGENDDGNNEDNEKLRKPKHKKLKQPADGNPDP (SEQ ID NO: 1), or wherein the peptide comprises SEQ ID NO:25 and N-terminal or C-terminal contiguous amino acids from the CSP NT sequences, SEQ ID NO: 57 or SEQ ID NO: 1, or a corresponding sequence from a variant strain.

17. A pharmaceutical composition of claim 13, further comprising an antigen peptide or a sequence encoding a peptide antigen representing NANP and or CT subregions of PfCSP and presenting NANP and/or CT epitopes of PfCSP.

18. A viral like particle comprising an antigen as defined in claim 13.

19. A vector or polynucleotide encoding and capable of expressing an antigen as defined in claim 13.

20. A viral or non-viral vector comprising a nucleic acid sequence encoding a peptide antigen representing or comprising NT epitopes of PfCSP and optionally substantially not representing or comprising CSP CT and/or NANP epitopes.

21. A method of treating or preventing malaria comprising administering to a subject an effective amount of a composition as defined in claim 13.

22.-23. (canceled)

24. The method of claim 1, wherein the CSP NT peptides are PCMS antigens or epitopes that promote in a subject antibody dependent phagocytosis or complement mediated killing of Sporozoites (PCMS).


SEQUENCE LISTING (60 sequences)

SEQ ID NO: 1 - protein, 47 aa, Artificial
  Malaria
KQENWYSLKKNSRSLGENDDGNNEDNEKLRKPKHKKLKQPADGNPDP

SEQ ID NO: 2 - protein, 24 aa, Artificial
  Malaria
KQENWYSLKKNSRSLGENDDGNNE

SEQ ID NO: 3 - protein, 21 aa, Artificial
  Malaria
SLKKNSRSLGENDDGNNEDNE

SEQ ID NO: 4 - protein, 23 aa, Artificial
  Malaria
DNEKLRKPKHKKLKQPADGNPDP

SEQ ID NO: 5 - protein, 25 aa, Artificial
  Malaria
DDGNNEDNEKLRKPKHKKLKQPADG

SEQ ID NO: 6 - protein, 3 aa, Artificial
  Malaria
DDG

SEQ ID NO: 7 - protein, 3 aa, Artificial
  Malaria
GNN

SEQ ID NO: 8 - protein, 4 aa, Artificial
  Malaria
DDGN

SEQ ID NO: 9 - protein, 5 aa, Artificial
  Malaria
DDGNN

SEQ ID NO: 10 - protein, 6 aa, Artificial
  Malaria
DDGNNE

SEQ ID NO: 11 - protein, 7 aa, Artificial
  Malaria
DDGNNED

SEQ ID NO: 12 - protein, 8 aa, Artificial
  Malaria
DDGNNEDN

SEQ ID NO: 13 - protein, 9 aa, Artificial
  Malaria
DDGNNEDNE

SEQ ID NO: 14 - protein, 10 aa, Artificial
  Malaria
DDGNNEDNEK

SEQ ID NO: 15 - protein, 11 aa, Artificial
  Malaria
DDGNNEDNEKL

SEQ ID NO: 16 - protein, 12 aa, Artificial
  Malaria
DDGNNEDNEKLR

SEQ ID NO: 17 - protein, 13 aa, Artificial
  Malaria
DDGNNEDNEKLRK

SEQ ID NO: 18 - protein, 14 aa, Artificial
  Malaria
DDGNNEDNEKLRKP

SEQ ID NO: 19 - protein, 15 aa, Artificial
  Malaria
DDGNNEDNEKLRKPK

SEQ ID NO: 20 - protein, 16 aa, Artificial
  Malaria
DDGNNEDNEKLRKPKH

SEQ ID NO: 21 - protein, 17 aa, Artificial
  Malaria
DDGNNEDNEKLRKPKHK

SEQ ID NO: 22 - protein, 18 aa, Artificial
  Malaria
DDGNNEDNEKLRKPKHKK

SEQ ID NO: 23 - protein, 19 aa, Artificial
  Malaria
DDGNNEDNEKLRKPKHKKL

SEQ ID NO: 24 - protein, 20 aa, Artificial
  Malaria
DDGNNEDNEKLRKPKHKKLK

SEQ ID NO: 25 - protein, 21 aa, Artificial
  Malaria
DDGNNEDNEKLRKPKHKKLKQ

SEQ ID NO: 26 - protein, 22 aa, Artificial
  Malaria
DDGNNEDNEKLRKPKHKKLKQP

SEQ ID NO: 27 - protein, 23 aa, Artificial
  Malaria
DDGNNEDNEKLRKPKHKKLKQPA

SEQ ID NO: 28 - protein, 24 aa, Artificial
  Malaria
DDGNNEDNEKLRKPKHKKLKQPAD

SEQ ID NO: 29 - protein, 23 aa, Artificial
  Malaria
KQENWYSLKKNSRSLGENDDGNN

SEQ ID NO: 30 - protein, 22 aa, Artificial
  Malaria
QENWYSLKKNSRSLGENDDGNN

SEQ ID NO: 31 - protein, 14 aa, Artificial
  Malaria
KNSRSLGENDDGNN

SEQ ID NO: 32 - protein, 13 aa, Artificial
  Malaria
NSRSLGENDDGNN

SEQ ID NO: 33 - protein, 11 aa, Artificial
  Malaria
RSLGENDDGNN

SEQ ID NO: 34 - protein, 10 aa, Artificial
  Malaria
SLGENDDGNN

SEQ ID NO: 35 - protein, 9 aa, Artificial
  Malaria
LGENDDGNN

SEQ ID NO: 36 - protein, 8 aa, Artificial
  Malaria
GENDDGNN

SEQ ID NO: 37 - protein, 23 aa, Artificial
  Malaria
KQENWYSLKKNSRSLGENDDGNN

SEQ ID NO: 38 - protein, 7 aa, Artificial
  Malaria
ENDDGNN

SEQ ID NO: 39 - protein, 6 aa, Artificial
  Malaria
NDDGNN

SEQ ID NO: 40 - protein, 3 aa, Artificial
  Malaria
DDG

SEQ ID NO: 41 - protein, 327 aa, Artificial
  Malaria
QENWYSLKKNSRSLGENDDGNNEDNEKLRKPKHKKLKQPADGNPDPNANPNVDPNANPNVDPNANPNVDPNANPNANPNANPNANPNANPNANPNANPNANPNANPNANPNANPNANPNANPNANPNANPNANPNANPNVDPNANPNANPNANPNANPNANPNANPNANPNANPNANPNANPNANPNANPNANPNANPNANPNANPNANPNANPNKNNQGNGQGHNMPNDPNRNVDENANANSAVKNNNNEEPSDKHIKEYLNKIQNSLSTEWSPCSVTCGNGIQVRIKPGSANKPKDELDYANDIEKKICKMEKCSSVFNVVNSGS

SEQ ID NO: 42 - protein, 15 aa, Artificial
  Malaria
KQENWYSLKKNSRSL

SEQ ID NO: 43 - protein, 15 aa, Artificial
  Malaria
NWYSLKKNSRSLGEN

SEQ ID NO: 44 - protein, 15 aa, Artificial
  Malaria
SLKKNSRSLGENDDG

SEQ ID NO: 45 - protein, 15 aa, Artificial
  Malaria
KNSRSLGENDDGNNE

SEQ ID NO: 46 - protein, 15 aa, Artificial
  Malaria
RSLGENDDGNNEDNE

SEQ ID NO: 47 - protein, 15 aa, Artificial
  Malaria
GENDDGNNEDNEKLR

SEQ ID NO: 48 - protein, 15 aa, Artificial
  Malaria
DDGNNEDNEKLRKPK

SEQ ID NO: 49 - protein, 15 aa, Artificial
  Malaria
NNEDNEKLRKPKHKK

SEQ ID NO: 50 - protein, 15 aa, Artificial
  Malaria
DNEKLRKPKHKKLKQ

SEQ ID NO: 51 - protein, 15 aa, Artificial
  Malaria
KLRKPKHKKLKQPAD

SEQ ID NO: 52 - protein, 15 aa, Artificial
  Malaria
KPKHKKLKQPADGNP

SEQ ID NO: 53 - protein, 15 aa, Artificial
  Malaria
HKKLKQPADGNPDPN

SEQ ID NO: 54 - protein, 15 aa, Artificial
  Malaria
LKQPADGNPDPNANP

SEQ ID NO: 55 - protein, 397 aa, Artificial
  Malaria
MMRKLAILSVSSFLFVEALFQEYQCYGSSSNTRVLNELNYDNAGTNLYNELEMNYYGKQENWYSLKKNSRSLGENDDGNNEDNEKLRKPKHKKLKQPADGNPDPNANPNVDPNANPNVDPNANPNVDPNANPNANPNANPNANPNANPNANPNANPNANPNANPNANPNANPNANPNANPNANPNANPNANPNANPNVDPNANPNANPNANPNANPNANPNANPNANPNANPNANPNANPNANPNANPNANPNANPNANPNANPNANPNANPNKNNQGNGQGHNMPNDPNRNVDENANANSAVKNNNNEEPSDKHIKEYLNKIQNSLSTEWSPCSVTCGNGIQVRIKPGSANKPKDELDYANDIEKKICKMEKCSSVFNVVNSSIGLIMVLSFLFLN

SEQ ID NO: 56 - DNA, 1195 bp, Artificial
  Malaria
atgatgagaaaattagctattttatctgtttcttcctttttatttgttgaggccttattccaggaataccagtgctatggaagttcgtcaaacacaagggttctaaatgaattaaattatgataatgcaggcactaatttatataatgaattagaaatgaattattatgggaaacaggaaaattggtatagtcttaaaaaaaatagtagatcacttggagaaaatgatgatggaaataacgaagacaacgagaaattaaggaaaccaaaacataaaaaattaaagcaaccagcggatggtaatcctgatccaaatgcaaacccaaatgtagatcccaatgccaacccaaatgtagatccaaatgcaaacccaaatgtagatccaaatgcaaacccaaatgcaaacccaaatgcaaacccaaatgcaaacccaaatgcaaacccaaatgcaaacccaaatgcaaacccaaatgcaaacccaaatgcaaacccaaatgcaaacccaaatgcaaacccaaatgcaaacccaaatgcaaacccaaatgcaaaccccaatgcaaatcctaatgcaaacccaaatgcaaacccaaacgtagatcctaatgcaaatccaaatgcaaacccaaacgcaaaccccaatgcaaatcctaatgcaaaccccaatgcaaatcctaatgcaaatcctaatgccaatccaaatgcaaatccaaatgcaaacccaaacgcaaaccccaatgcaaatcctaatgccaatccaaatgcaaatccaaatgcaaacccaaatgcaaacccaaatgcaaaccccaatgcaaatcctaataaaaacaatcaaggtaatggacaaggtcacaatatgccaaatgacccaaaccgaaatgtagatgaaaatgctaatgccaacagtgctgtaaaaaataataataacgaagaaccaagtgataagcacataaaagaatatttaaacaaaatacaaaattctctttcaactgaatggtccccatgtagtgtaacttgtggaaatggtattcaagttagaataaagcctggctctgctaataaacctaaagacgaattagattatgcaaatgatattgaaaaaaaaatttgtaaaatggaaaaatgttccagtgtgtttaatgtcgtaaatagttcaataggattaataatggtattatccttcttgttccttaattagg

SEQ ID NO: 57 - protein, 41 aa, Artificial
  NT peptide
ENWYSLKKNSRSLGENDDGNNEDNEKLRKPKHKKLKQPADG

SEQ ID NO: 58 - protein, 15 aa, Artificial
  T cell helper epitope
QYIKANSKFIGITEL

SEQ ID NO: 59 - protein, 4 aa, Artificial
  spacer
SGSG

SEQ ID NO: 60 - protein, 59 aa, Artificial
  PCMS antigen
ENWYSLKKNSRSLGENDDGNNEDNEKLRKPKHKKLKQPADSGSGQYIKANSKFIGITEL